(19) United States
(12) Patent Application Publication
(10) Pub. No.: US 2026/0295058 A1
(43) Pub. Date: Oct. 1, 2026

(54) PHLIP®-MEDIATED DELIVERY OF STING AGONISTS

(71) Applicants: UNIVERSITY OF RHODE ISLAND BOARD OF TRUSTEES, Kingston, RI (US); YALE UNIVERSITY, New Haven, CT (US)

(72) Inventors: Yana K. RESHETNYAK, Saunderstown, RI (US); Oleg A. ANDREEV, Saunderstown, RI (US); Donald M. ENGELMAN, New Haven, CT (US); Anna MOSHNIKOVA, Warwick, RI (US)

(21) Appl. No.: 19/102,302

(22) PCT Filed: Aug. 14, 2023
(86) PCT No.: PCT/US2023/072169

§ 371 Date: Feb. 7, 2025

Related U.S. Application Data
(60) Provisional application No. 63/371,486, filed on Aug. 15, 2022.

(51) Int. Cl. A61K 47/64; A61K 47/54; A61P 35/00

(52) U.S. Cl. CPC A61K 47/64; A61K 47/545; A61P 35/00

(57) ABSTRACT

Compositions comprising a pHLIP® peptide linked to a STING agonist, pharmaceutical compositions thereof, and methods of treatment using such compositions are disclosed herein.

### CROSS-REFERENCE TO RELATED APPLICATIONS

This patent application is a National Stage Entry of International Application PCT/US2023/072169, filed on Aug. 14, 2013, which claims the priority benefit of U.S. Provisional Application No. 63/371,486, filed on Aug. 15, 2022, the disclosure of which is hereby incorporated by reference herein, in its entirety.

### STATEMENT AS TO FEDERALLY SPONSORED RESEARCH

This invention was made with government support under grant number R01 GM073857 awarded by the National Institutes of Health. The government has certain rights in the invention.

### REFERENCE TO A SEQUENCE LISTING SUBMITTED ELECTRONICALLY

The content of the electronically submitted sequence listing (Name: 5095_0130001_SequenceListing_ST26.xml; Size: 212,987 bytes; and Date of Creation: Jan. 20, 2026) is herein incorporated by reference in its entirety.

### FIELD OF THE INVENTION

Compositions comprising a pHLIP® peptide linked to a STING agonist, pharmaceutical compositions thereof, and methods of treatment using such compositions are disclosed herein.

### BACKGROUND

Conventional methods of cancer treatment employ toxic agents that do not distinguish between normal and tumor tissues very well, and are therefore limited in their use because of their harmful side effects. Recent studies in cancer immunotherapy have shown that boosting the innate immune system of patients with cancer can have a critical effect in the outcome of cancer treatment, alone or in combination with conventional treatments. Stimulator of interferon genes (STING) agonists have targets localized in the cytosol of a tumor cell including cancer cells and activated myeloid cells within tumor microenvironment. Although STING agonists (STINGa) stimulate an immune response, it is necessary to specifically and efficiently target tumor cells to avoid adverse side effects. There is also a significant advantage in delivering it to the activated immune cells within tumors, which leads to more potent activation of the STING pathway and inflammatory response.

However, one obstacle in the use of small molecule STINGa is their poor pharmacokinetics. Another limitation is that some small molecule STINGa tend to be polar molecules that do not efficiently cross cell membranes. STING agonists, such as cyclic guanosine monophosphate-adenosine monophosphate (cGAMP) for instance, are hydrophilic and negatively charged molecules. Because cell membranes are non-polar, hydrophobic environments, such molecules are associated with poor membrane permeability.

Thus, a current challenge in the field of immunotherapy is to find methods to deliver STING agonists specifically into the cytosol to tumor cells, specifically to activated immune cells and potentially stroma cells, while avoiding delivery into healthy cells, and improve pharmacokinetics of small molecule STINGa.

### SUMMARY OF THE INVENTION

Compositions comprising the following structure:

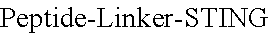

wherein Peptide is a pHLIP® peptide,
wherein Linker is a cleavable linker;
wherein “STING” is a STING agonist selected from the group consisting of diABZI, ADU-S100 (MIW815), MK-1454, MK-2118, SB11285, GSK3745417, BMS-986301, BI1387446, E7766, TAK-676, SNZ281, IMSA-101, IMSA-201, SYNB1891, JNJ-4412, JNJ-6196, BI-STING, GSK532, TTI-10001, CRD5500, LB-061, ALG-031048, RVU312-4787, TAK-500, CL656, CDK-002, CF501, CF502, CF503, CF504, CF505, CF506, CF507, CF508, CF509, CF510, CF511, and derivatives thereof; and wherein each “-” is a covalent bond are described herein.

In some aspects, the pHLIP® peptide has the following sequence: XₙYₘ; YₘXₙ; XₙYₘXⱼ; YₘXₙYᵢ; YₘXₙYᵢXⱼ; XₙYₘXⱼYᵢ; YₘXₙYᵢXⱼYₗ; XₙYₘXⱼYᵢXₗ; YₘXₙYᵢXⱼYₗXₕ; XₙYₘXⱼYᵢXₕY_{g}; YₘXₙYᵢXⱼYₗXₕ Y_{g}; XₙYₘXⱼYᵢXₕY_{g}X_{f}; (XY)ₙ; (YX)ₙ; (XY)ₙ Yₘ; (YX)ₙYₘ; (XY)ₙXₘ; (YX)ₙXₘ; Yₘ(XY)ₙ; Yₘ (YX)ₙ; Xₙ(XY)ₘ; Xₙ(YX)ₘ; (XY)ₙYₘ (XY)ᵢ; (YX)ₙYₘ (YX)ᵢ; (XY)ₙXₘ(XY)ᵢ; (YX)ₙXₘ(YX)ᵢ; Yₘ (XY)ₙ; Yₘ (YX)ₙ; Xₙ(XY)ₘ; or Xₙ(YX)ₘ; wherein,
(i) each Y is, independently, a non-polar amino acid with solvation energy, DG_{X}^{cor}>+0.50, or Glycine (Gly);
(ii) each X is, independently, a protonatable amino acid,
(iii) n, m, i, j, 1, h, g, f are each, independently, an integer from 1 to 8.

In some aspects, the pHILIP® peptide comprises a sequence comprising the sequence ADDQNPWRAYLDLLFPTDTLLLDLLWXG (SEQ ID NO: 220), wherein “X” is a functional group for conjugation purposes, selected from lysine (Lys), cysteine (Cys), Azido-containing amino acid or other modified amino acids. In some aspects, the pHLIP® peptide comprises a sequence consisting of the sequence ADDQNPWRAYLDLLFPTDTLLLDLLWXG (SEQ ID NO: 220), wherein “X” is a functional group for conjugation purposes, selected from lysine (Lys), cysteine (Cys), Azido-containing amino acid or other modified amino acids.

In some aspects, the pHLIP® peptide comprises a sequence selected from the group consisting of any one of SEQ ID NOs. 1-219. In some aspects, the pHLIP® peptide has a sequence selected from the group consisting of any one of SEQ ID NOs. 1-219. In some aspects, the pHLIP® peptide has a sequence comprising the sequence ADQDNPWRAYLDLLFPTDTLLLDLLWCG (SEQ ID NO: 218). In some aspects, the pHLIP® peptide has a sequence comprising the sequence ADDQNPWRAYLDLLFPTDTLLLDLLWCG (SEQ ID NO: 219). In some aspects, the pHLIP® peptide has a sequence consisting of the sequence ADQDNPWRAYLDLLFPTDTLLLDLLWCG (SEQ ID NO: 218). In some aspects, the pHLIP® peptide has a sequence consisting of the sequence

(SEQ ID NO: 219)
ADDQNPWRAYLDLLFPTDTLLLDLLWCG.

Compositions comprising the following structure:

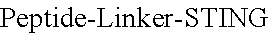

wherein Peptide is a pHLIP® peptide comprising the sequence

(SEQ ID NO: 218)
ADQDNPWRAYLDLLFPTDTLLLDLLWCG
or
(SEQ ID NO: 219)
ADDQNPWRAYLDLLFPTDTLLLDLLWCG;

wherein Linker is a cleavable linker
wherein STING is a STING agonist; and
wherein each “-” is a covalent bond.

In some aspects, the pHLIP® peptide comprises the sequence ADQDNPWRAYLDLLFPTDTLLLDLLWCG (SEQ ID NO: 218). In some aspects, the pHLIP® peptide comprises the sequence ADDQNPWRAYLDLLFPTDTLLLDLLWCG (SEQ ID NO: 219). In some aspects, the pHLIP® peptide consists of the sequence ADQDNPWRAYLDLLFPTDTLLLDLLWCG (SEQ ID NO: 218) or ADDQNPWRAYLDLLFPTDTLLLDLLWCG (SEQ ID NO: 219). In some aspects, the pHLIP® peptide consists of the sequence ADQDNPWRAYLDLLFPTDTLLLDLLWCG (SEQ ID NO: 218). In some aspects, the pHLIP® peptide consists of the sequence

(SEQ ID NO: 219)
ADDQNPWRAYLDLLFPTDTLLLDLLWCG.

In some aspects, the STING agonist comprises a cyclic dinucleotide (CDN). In other aspects, the STING agonist comprises a cyclic purine dinucleotide. In some aspects, the STING agonist is a non-nucleotide small molecule. In some aspects, the STING agonist is selected from the group consisting of diABZI, ADU-S100 (MIW815), MK-1454, MK-2118, SB11285, GSK3745417, BMS-986301, BI1387446, E7766, TAK-676, SNZ281, IMSA-101, IMSA-201, SYNB1891, JNJ-4412, JNJ-6196, BI-STING, GSK532, TTI-10001, CRD5500, LB-061, ALG-031048, RVU312-4787, TAK-500, CL656, CDK-002, CF501, CF502, CF503, CF504, CF505, CF506, CF507, CF508, CF509, CF510, CF511, and derivatives thereof. In some aspects, the STING agonist is diABZI. In some aspects, the STING agonist is MK-1454. In some aspects, the STING agonist is MK-2118. In some aspect, the STING agonist is TAK-676. In other aspects, the STING agonist is BMS-986301. In some aspect, the STING agonist is BI1387446.

In some aspects, all amino acids in the pHLIP® peptide of the compositions disclosed herein are D-amino acids.

In some aspects, the Linker of the compositions disclosed herein comprise a disulfide bond or an acid-labile bond. In some aspects, the Linker is self-immolating.

In some aspects, the composition has the following structure:

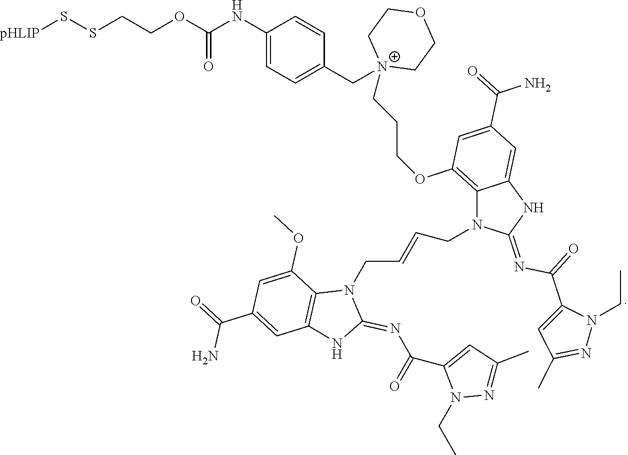

In some aspects, pharmaceutical compositions comprising at least one of the compositions disclosed herein and one or more pharmaceutically excipients are provided.

In some aspects, methods of treating cancer in a subject in need thereof comprising administering a composition or a pharmaceutical composition disclosed herein to the subject are provided. In some aspects, the cancer is a solid tumor. In some aspects, a composition or pharmaceutical composition disclosed herein is injected directly into a tumor mass. In some aspects, a composition or pharmaceutical composition disclosed herein is administered systemically. In some aspects, the subject is a human.

### Definitions

A pHLIP® peptide (pH Low Insertion Peptide) is a linear, water-soluble membrane peptide that interacts weakly with a cell membrane at neutral pH, without insertion into the lipid bilayer; however, at slightly acidic pH (<7.0), pHLIP® inserts into the cell membrane and forms a stable transmembrane alpha-helix. In addition to tumor cells characterized by low pH (<7.0), immune cells within a tumor mass are also characterized by low pH (<7.0). For example, the cells within the environment of a tumor mass, e.g., macrophages, are also characterized by low pH. By binding a pHLIP® to a STING agonist, the compositions and methods specifically deliver the drug directly to the cancerous and immune (macrophages) cells and into their cytosols due to their acidic cell surfaces. Delivering a STING agonist using pHLIP® peptides boosts the immune system, e.g., by stimulating the production of interferon-gamma, to mount and/or establish a tumor specific immune response and fight the cancer.

As used herein, “effective,” when referring to an amount of a composition, refers to the quantity of the composition that is sufficient to yield a desired response. For example, the amount of cargo, e.g., STING agonist yields a desired response, e.g., augmentation of an anti-tumor effect, without undue adverse side effects (such as toxicity, irritation, or allergic response) commensurate with a reasonable benefit/risk ratio when used in the manner of this disclosure.

In some embodiments, a subject is a mammal. In certain embodiments, the mammal is a rodent (e.g., a mouse or a rat), a primate (e.g., a chimpanzee, a gorilla, a monkey, a gibbon, a baboon), a cow, a camel, a dog, a cat, a horse, a llama, a sheep, a goat, or a pig. In some embodiments, the subject is a human.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, “treating” encompasses, e.g., inhibition, regression, or stasis of the progression of a disorder. Treating also encompasses the prevention or amelioration of any symptom or symptoms of the disorder. As used herein, “inhibition” of disease progression or a disease complication in a subject means preventing or reducing the disease progression and/or disease complication in the subject.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

Each embodiment disclosed herein is contemplated as being applicable to each of the other disclosed embodiments. Thus, all combinations of the various elements described herein are within the scope of the invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### DESCRIPTION OF THE DRAWINGS

FIG. 1. Characterization of pHLIP-STINGa a) Schematic presentation of pHLIP-STINGa interaction with a membrane lipid bilayer (the pHILIP polymer is shown in dark blue and STINGa is shown by a red circle). In state I, pHLIP-STINGa forms an unstructured polymer in solution at normal pH. State II shows the interaction of pHLIP-STINGa with the membrane at normal pH. State III represents the transmembrane helical orientation of pHLIP triggered by low pH, which leads to the translocation of STINGa across the lipid bilayer and its release in the cytoplasm. The three states were monitored by changes of fluorescence (b) and CD (c) spectral signals of pHLIP-STINGa interacting with POPC liposomes. d) Kinetics of fluorescence changes triggered by pH drop in presence of POPC liposomes is shown. pH transitions monitored by changes of fluorescence intensity (e) and CD (f) spectral signals are shown (experimental points and fitting curves, red, with 95% confidence interval, pink). g) OCD spectra of pHLIP-STINGa recorded immediately after deposition of pHLIP-STINGa on the supported bilayer and 12 hrs later, when the insertion of pHLIP-STINGa into bilayer was complete, are shown. h) Activation of the IFN signaling pathway induced by pHLIP-STINGa in THP1-Blue-ISG cells polarized by PMA, IL-4/IL-13 into M2-type macrophages is shown. The results were normalized to the activity of STINGa alone at the maximum concentration tested, which was taken as 100%.

FIG. 2. Tumor targeting, PK, biodistribution, tumor and serum cytokines, tumor stroma and immune cell uptake. a) Imaging of CT26 tumor targeting in mouse performed at 40 hrs after single IP injection of ICG-pHLIP-STINGa (100 μM 300 μl). Tumor site prior to and after shaving is shown. b) Normalized fluorescence recorded in blood, which was collected at different time points after single IV injection of ICG-pHLIP-STINGa (200 μM 100 μl), is shown (mean and SE). The data were fitted by exponential function (red line). c) Kinetics of ICG-pHLIP-STINGa targeting of CT26 tumor and clearance of the agent from major organs are shown. The mean fluorescence per area was calculated for each organ and tissue collected at different timepoints after single IV injections of ICG-pHLIP-STINGa (200 μM 100 μl). d) Level of IL-6 and TNF-α cytokines in tumors and serum established by ELISA at different time points after a single IV injection of STINGa or pHLIP-STINGa (200 μM 150 μl) in comparison to control mice are shown (all points, mean and SE are shown, p-levels were calculated using the Kolmogorov-Smirnov two-tailed nonparametric test). e) Percent of populations of tumor stroma and immune cells within TME targeted by A1647-pHLIP-STINGa was established by FACS analysis on CT26 tumors collected 24 hrs after a single IP injection of A1647-pHLIP-STINGa (100 μM 300 μl) (all points, mean, St.d. are shown on graphs and numbers are given in the table). f) Co-localization of A1647-pHLIP-STINGa with A1488-CD140b-Antb staining CAFs, A1594-CD206-Antb staining TAMs and DAPI staining cell nuclei within TME are shown on images obtained at different magnifications using 10×, 20× and 40× objectives.

FIG. 3. Eradication of CT26 tumors and development of immune memory. a) Experimental design. b) CT26 tumor growth curves in Balb/c mice are shown after a single administration of different agents at dose levels of 100 μM 300 μl for IP injections or 200 μM 150 μl for IV injections performed on day 1, when tumors had reached about 100 mm³ in volume. The star (*) indicates that this mouse in IV group received a second dose of pHLIP-STINGa on day 41, when the tumor had started to re-grow. c) Kaplan-Meier survival plots obtained for the data shown in panel b. d) CT26 tumor growth curves obtained for tumor-free mice from the groups that received single IP or IV injections of pHLIP-STINGa on day 1. On day 61 CT26 cancer cells were re-injected in the left flanks of these mice. e) CT26 large tumor growth curves are shown after a single IP injection of pHLIP-STINGa (100 μM 300 μl) on day 1, when tumors had reached 400-700 mm³ in volume.

FIG. 4. Obliteration of tumor stroma and pH increase within the TME. a-d) The numbers of cells within the TME in control (mice receiving vehicle as a single IP injection) and treated (mice receiving a single IP injection of A1647-pHLIP-STINGa (100 μM 300 μl)) were established by FACS analysis. The percentages of all live cells with TME (a), the number of CAFs (CD3⁺, CD45⁺, CD140b⁺) quantified as % of all live cells (b), the numbers of TAMs (CD45⁺, CD11b⁺, F4/80⁺, CD206⁺) (c) and mMDSCs (CD45⁺, CD3⁻, CD11b⁺, F4/80⁻, Ly6C^{hi}, Ly6G⁻) (d) both quantified as % of CD45⁺ immune cells are shown (all points, mean and St.D., the p-levels for all graphs<0.002). e-g) pH imaging with ICG-pHLIP and quantification of the signal are shown. Mice bearing CT26 tumor in right flank (about 100 mm³) were divided into 2 groups (5 animals per group). One group (treated) was treated with a single IP injection of pHLIP-STINGa (100 μM 300 μl) on day 1 and another group (control) did not receive any treatment. On day 3 an ICG-pHLIP pH-imaging probe was given in single IP injections (50 μM 100 μl) to both control (non-treated) and pHLIP-STINGa treated groups, and 24 hrs later (on day 4), animals were euthanized, tumors were collected, cut in half and imaged, the representative images are shown on panels e-f, and normalized mean fluorescence signal calculated for all tumor pieces (5 animals per group) are shown on panel g (all points, mean and St.D. are shown, p-level was calculated using the Kolmogorov-Smirnov two-tailed nonparametric test).

FIG. 5. Kinetics of self-immolation. HPLC chromatograms recorded at 280 nm and 320 nm for pHLIP-STINGa treated with DTT dissolved in 50 mM phosphate buffer containing 150 mM NaCl pH 7.4 at different time points (0, 30, 60 min) after addition of DTT are shown. Retention times are the following pHLIP-STINGa: 16.78 min, pHLIP: 17.02 min (has no signal at 320 nm), SH-PAB-STINGa (modified diABZI cleaved from pHLIP): 12.16 min, STINGa (original diABZI): 10.27 min, product of immolation: 6.95 min.

FIG. 6. DiABZI fluorescence. Fluorescence spectra of STINGa (diABZI) in phosphate buffer excited at 295 nm and 322 nm are shown.

FIG. 7. OCD spectra. OCD spectra were recorded 12 hours after deposition of solution of pHLIP-STINGa and POPC in supported layers at pH 5 and pH 3.3.

FIG. 8. Cell viability. THP1 cells were activated and polarized into M2 macrophages by treatment with PMA for 6 hours followed by treatment with IL-4/IL-13 for another 18 hours. After activation and polarization all factors were removed and cells were treated with pHLIP-STINGa at pH 6.4 in DMEM without FBS for 3 hours. Then, FBS was added up to 10% and pH increased to pH 7.4 and cells were incubated for 48 hours after which MTS calorimetric assay was performed.

FIG. 9. Biodistribution of ICG-pHLIP-STINGa in organs and tissues. Mean fluorescence per area was calculated for each tissue and organ at 2, 4, 24, 48, 72 and 96 hours post-injection after a single IV injection of ICG-pHLIP-STINGa (200 μM 100 μl). All points, means and St.d. are shown.

FIG. 10. Average level of cytokines (IL-6, TNF-α, IFN-β) in tumor supernatants and serum measured by ELISA at 4 and 16 hrs after a single IV (200 μM 150 μl) or IP (100 μM 300 μl) injections of pHLIP(Laa)-STINGa and pHLIP(Daa)-STINGa.

FIG. 11. Immune memory was not developed for 4T1 cancer cells. 4T1 tumors growth curves in “control” group (a) and “second time re-injected with 4T1 cancer cells” group (b) are shown. Mice in control group received 10⁵ 4T1 cancer cells in right flank. Mice in the second time re-injected group were first inoculated with 5×10⁴ CT26 cancer cells. When tumors reached about 100 mm³ in volume mice received a single IP injection of pHLIP-STINGa (100 μM 300 μl) on day 1, tumors were eradicated, then on day 61, 5×10⁴ CT26 cancer cells were inoculated in left flank, tumors did not develop, and, finally, on day 101 10⁵ 4T1 cancer cells were inoculated in right flank and 4T1 tumors were developed. c) Kaplan-Meier survival plots obtained for data shown on panels a and b.

FIG. 12. Treatment of CT26 tumors in athymic nude mice lacking T-cells. a-b) CT26 tumors growth curves in nude athymic mice are shown after a single IP injection of pHLIP-STINGa (100 μM 300 μl) on day 1, when tumors reached about 100-200 mm³ in volume. c) Kaplan-Meier survival plots obtained for data shown on panels a-b.

FIG. 13. Treatment of 4T1 tumors. 10⁵ of 4T1 breast cancer cells were inoculated in right flank of Balb/C mice. When tumor reached about 100 mm³ in volume (day 1) a single IP injection of pHLIP-STINGa (100 μM 300 μl) was given to groups pHLIP-STINGa and pHLIP-STINGa+PD-1 (shown by red arrow), and 3 IP injections on days 4, 9 and 14 of PD-1antibody (250 μg per injection) were given to groups PD-1 and pHLIP-STINGa+PD-1 (shown by blue arrow). Control group did not receive treatment. Tumors growth of control (a), pHLIP-STINGa (b), PD-1 (c) and pHLIP-STINGa+PD-1 (d) are shown. e) Kaplan-Meier survival plots based on the data shown on panels a-d are presented. The p-levels for statistical significance of improved survival of pHLIP-STINGa vs control and PD-1 vs control was established to be p-level<0.02 calculated using Log rank (by weighting all time points the same), Breslow method (by weighting all time points by the number of cases at risk at each time point) and Tarone-Ware method (by weighting all time points by the square root of the number of cases at risk at each time point), and p<0.004 for pHLIP-STINGa+PD-1 vs control.

FIG. 14. Body weight. The change of body weight after a single IP injection of pHLIP-STINGa (100 μM 300 μl) received on day 1 is shown. Similar body weight curves were obtained for other routes of administrations in Balb/c and athymic nude mice.

FIG. 15. Intra-tumoral hemorrhage was observed within 2-4 days after pHLIP-STINGa injection. Image of Balb/c mice with CT26 tumor in right flank next day after a single IP injection of pHLIP-STINGa (300 μM 100 μl) is shown.

### DETAILED DESCRIPTION

Compositions comprising a pHLIP® peptide linked to a STING agonists are described herein.

### STING Agonists

Cyclic dinucleotides are exemplary STING agonists, but other small molecules can be used as well. Exemplary STING agonists include diABZI, ADU-S100 (MIW815), MK-1454, MK-2118, SB11285, GSK3745417, BMS-986301, BI1387446, E7766, TAK-676, SNZ281, IMSA-101, IMSA-201, SYNB1891, JNJ-4412, JNJ-6196, BI-STING, GSK532, TTI-10001, CRD5500, LB-061, ALG-031048, RVU312-4787, TAK-500, CL656, CDK-002, CF501, CF502, CF503, CF504, CF505, CF506, CF507, CF508, CF509, CF510, CF511, and derivatives thereof. In some aspects, the STING agonist is selected from the group consisting of diABZI, ADU-S100 (MIW815), MK-1454, MK-2118, SB11285, GSK3745417, BMS-986301, BI1387446, E7766, TAK-676, SNZ281, IMSA-101, IMSA-201, SYNB1891, JNJ-4412, JNJ-6196, BI-STING, GSK532, TTI-10001, CRD5500, LB-061, ALG-031048, RVU312-4787, TAK-500, CL656, CDK-002, CF501, CF502, CF503, CF504, CF505, CF506, CF507, CF508, CF509, CF510, CF511, and derivatives thereof. In some aspects, the STING agonist is selected from the group consisting of diABZI, ADU-S100 (MIW815), MK-1454, MK-2118, SB11285, GSK3745417, BMS-986301, BI1387446, E7766, TAK-676, SNZ281, IMSA-101, IMSA-201, SYNB1891, JNJ-4412, JNJ-6196, BI-STING, GSK532, TTI-10001, CRD5500, LB-061, ALG-031048, RVU312-4787, TAK-500, CL656, CDK-002, CF501, CF502, CF503, CF504, CF505, CF506, CF507, CF508, CF509, CF510, CF511. In one aspect, the STING agonist is diABZI. In some aspects, the STING agonist is MK-1454. In some aspects, the STING agonist is MK-2218.

pHLIP® Peptides

In some aspects, the pHLIP® peptide has the following sequence: XₙYₘ; YₘXₙ; XₙYₘXⱼ; YₘXₙYᵢ; YₘXₙYᵢXⱼ; XₙYₘXⱼYᵢ; YₘXₙYᵢXⱼYₗ; XₙYₘXⱼYᵢXₗ; YₘXₙYᵢXⱼYₗXₕ; XₙYₘXⱼYᵢXₕY_{g}; YₘXₙYᵢXⱼYₗXₕY_{g}; XₙYₘXⱼYᵢXₕY_{g}X_{f}; (XY)ₙ; (YX)ₙ; (XY)ₙ Yₘ; (YX)ₙYₘ; (XY)ₙXₘ; (YX)ₙXₘ; Yₘ (XY)ₙ; Yₘ (YX)ₙ; Xₙ(XY)ₘ; Xₙ(YX)ₘ; (XY)ₙYₘ (XY)ᵢ; (YX)ₙYₘ (YX)ᵢ; (XY)ₙXₘ(XY)ᵢ; (YX)ₙXₘ(YX)ᵢ; Yₘ (XY)ₙ; Yₘ (YX)ₙ; Xₙ(XY)ₘ; or Xₙ(YX)ₘ; wherein,
(i) each Y is, independently, a non-polar amino acid with solvation energy, DG_{X}^{cor}>+0.50, or Glycine (Gly);
(ii) each X is, independently, a protonatable amino acid,
(iii) n, m, i, j, 1, h, g, f are each, independently, an integer from 1 to 8.

In some aspects, the pHLIP® peptide comprises a sequence comprising the sequence ADDQNPWRAYLDLLFPTDTLLLDLLWXG (SEQ ID NO: 220), wherein “X” is a functional group for conjugation purposes, selected from lysine (Lys), cysteine (Cys), Azido-containing amino acid or other modified amino acids. In some aspects, the pHILIP® peptide comprises a sequence consisting of the sequence ADDQNPWRAYLDLLFPTDTLLLDLLWXG (SEQ ID NO: 220), wherein “X” is a functional group for conjugation purposes, selected from lysine (Lys), cysteine (Cys), Azido-containing amino acid or other modified amino acids.

An example of a pHLIP® peptide is AEQNPIYWARYADWLFTTPLLLLDLALLVDADEGT (SEQ ID NO: 3) in which AEQNPIY (SEQ ID NO: 4) represents a flanking sequence, WARYADWLFTTPLLLLDLALLV (SEQ ID NO: 5) represents a membrane-inserting sequence, and DADEGT (SEQ ID NO: 6) represents a flanking sequence

Other exemplary pHLIP® peptides are shown in the Tables below. The sequences are provided N-terminus to C-terminus.

TABLE 1
Non-limiting examples of pHLIP® sequences.

| Name | Sequence | SEQ ID NO: |
|---|---|---|
| Var3-1a | ADQDNPWRAYLDLLFPTDTLLLDLLWCA | SEQ ID NO: 212 |
| Var3-1b | ADQDNPWRAYLDLLFPTDTLLLDLLWKA | SEQ ID NO. 213 |
| WT-1 | GGEQNPIYWARYADWLFTTPLLLLDLALLVDADEGT | SEQ. ID NO. 7 |
| WT-2 | AEQNPIYWARYADWLFTTPLLLLDLALLVDADEGT | SEQ. ID NO. 8 |
| WT-Cys1 | AAEQNPIYWARYADWLFTTPLLLLDLALLVDADEGTCG | SEQ. ID NO. 9 |
| WT-Cys2 | Ac-AEQNPIYWARYADWLFTTPLLLLDLALLVDADEGCT | SEQ ID NO: 211 |
| WT-Cys3 | GGEQNPIYWARYADWLFTTPLLLLDLALLVDADEGTCG | SEQ. ID NO. 11 |
| Cys-WT1 | Ac-ACEQNPIYWARYADWLFTTPLLLLDLALLVDADEGTG | SEQ. ID NO. 12 |
| Var0-NT | ACEQNPIYWARYADWLFTTPLLLLDLALLVDADEGT | SEQ. ID NO. 13 |
| Lys-WT1 | AKEQNPIYWARYADWLFTTPLLLLDLALLVDADEGT | SEQ. ID NO. 14 |
| Lys-WT2 | Ac-AKEQNPIYWARYADWLFTTPLLLLDLALLVDADEGTG | SEQ ID NO: 15 |
| WT-KC | AAEQNPIYWARYADWLFTTPLLLLDLALLVDADEGTKCG | SEQ. ID NO. 16 |
| K-WT-C | AKEQNPIYWARYADWLFTTPLLLLDLALLVDADECT | SEQ. ID NO. 17 |
| N-pHLIP® | ACEQNPIYWARYANWLFTTPLLLLNLALLVDADEGTG | SEQ. ID NO. 18 |
| N-pHLIP®-b | ACEQNPIYWARYANWLFTTPLLLLNLALLVDADEGT | SEQ ID NO: 19 |
| K-pHLIP® | ACEQNPIYWARYAKWLFTTPLLLLKLALLVDADEGTG | SEQ. ID NO. 20 |
| NNQ | GGEQNPIYWARYADWLFTTPLLLLDLALLVNANQGT | SEQ. ID NO. 21 |
| D25A | AAEQNPIYWARYADWLFTTPLLLLALALLVDADEGT | SEQ. ID NO. 22 |
| D25A-KC | Ac-AAEQNPIYWARYADWLFTTPLLLLELALLVDADEGTKCG | SEQ ID NO: 23 |
| D14A | AAEQNPIYWARYAAWLFTTPLLLLDLALLVDADEGT | SEQ. ID NO. 24 |
| P20A | AAEQNPIYWARYADWLFTTALLLLDLALLVDADEGT | SEQ. ID NO. 25 |
| D25E | AAEQNPIYWARYADWLFTTPLLLLELALLVDADEGT | SEQ. ID NO. 26 |
| D14E | AAEQNPIYWARYAEWLFTTPLLLLDLALLVDADEGT | SEQ. ID NO. 27 |
| 3D | AAEQNPIIYWARYADWLFTDLPLLLLDLLALLVDADEGT | SEQ. ID NO. 28 |
| R11Q | GEQNPIYWAQYADWLFTTPLLLLDLALLVDADEGTCG | SEQ. ID NO. 29 |
| D25Up | GGEQNPIYWARYADWLFTTPLLLDLLALLVDADEGTCG | SEQ. ID NO. 30 |
| D25Down | GGEQNPIYWARYADWLFTTPLLLLLDALLVDADEGTCG | SEQ. ID NO. 31 |
| D14Up | GGEQNPIYWARYDAWLFTTPLLLLDLALLVDADEGTCG | SEQ. ID NO. 32 |
| D14Down | GGEQNPIYWARYAWDLFTTPLLLLDLALLVDADEGTCG | SEQ. ID NO. 33 |
| P20G | AAEQNPIYWARYADWLFTTGLLLLDLALLVDADEGT | SEQ. ID NO. 34 |
| H1-Cys | DDDEDNPIYWARYADWLFTTPLLLLHGALLVDADECT | SEQ. ID NO. 35 |
| H1 | DDDEDNPIYWARYADWLFTTPLLLLHGALLVDADET | SEQ ID NO: 36 |
| H2-Cys | DDDEDNPIYWARYAHWLFTTPLLLLHGALLVDADEGCT | SEQ. ID NO. 37 |
| Cys-H2 | CDDDEDNPIYWARYAHWLFTTPLLLLHGALLVDADET | SEQ ID NO: 38 |
| H2 | DDDEDNPIYWARYAHWLFTTPLLLLHGALLVDADEGT | SEQ ID NO: 39 |
| H2N-Cys | DDDEDNPIYWARYAHWLFTTPLLLLHGALLVNADECT | SEQ. ID NO. 40 |
| H2N | DDDEDNPIYWARYAHWLFTTPLLLLHGALLVNADEGT | SEQ ID NO: 41 |
| H2N2-Cys | DDDEDNPIYWARYAHWLFTTPLLLLHGALLVNANECT | SEQ. ID NO. 42 |
| H2N2 | DDDEDNPIYWARYAHWLFTTPLLLLHGALLVNANEGT | SEQ ID NO: 43 |
| 1a-Trp | AEQNPIYWARYADFLFTTPLLLLDLALLVDADET | SEQ. ID NO. 44 |
| 1b-Trp | AEQNPIYFARYADWLFTTPLLLLDLALLVDADEGT | SEQ. ID NO. 45 |
| 1c-Trp | AEQNPIYFARYADFLFTTPLLLLDLALLWDADET | SEQ. ID NO. 46 |
| Fast-1 or Var1 | AKEDQNPYWARYADWLFTTPLLLLDLALLVDG | SEQ. ID NO. 47 |
| Var1-2D1D | ACEDQNPYWARYADWLFTTPLLLLDLALLVDG | SEQ. ID NO. 48 |
| Fast1-Cys or | AEDQNPYWARYADWLFTTPLLLLDLALLVDCG | SEQ. ID NO. 49 |
| Var1-2DID-Cys | | |
| Fast1-E-Cys or | AEDQNPYWARYADWLFTTPLLLLELALLVECG | SEQ. ID NO. 50 |
| Var1E | | |
| Fast1-E-Lys | AKEDQNDPYWARYADWLFTTPLLLLDLALLVG | SEQ ID NO: 51 |
| Fast2 or Var2 | AKEDQNPYWRAYADLFTPLTLLDLLALWDG | SEQ. ID NO. 52 |
| Fast2-E-Cys or | AEDQNPYWARYADWLFTTPLLLLELALLVCG | SEQ ID NO: 53 |
| Var2E | | |
| Var2-2D1D | ACEDQNPYWRAYADLFTPLTLLDLLALWDG | SEQ. ID NO. 54 |
| Var3-3D | ACDDQNPWRAYLDLLFPTDTLLLDLLW | SEQ. ID NO. 55 |
| Var3-3D-cys | AKDDQNPWRAYLDLLFPTDTLLLDLLWC | SEQ ID NO: 56 |
| Var4-3E | ACEEQNPWRAYLELLFPTETLLLELLW | SEQ ID NO: 57 |
| Var5-3Da | ACDDQNPWARYLDWLFPTDTLLLDL | SEQ ID NO: 58 |
| Var6-3Db | CDNNNPWRAYLDLLFPTDTLLLDW | SEQ ID NO: 59 |
| Var8-3Eb | CEEQQPWAQYLELLFPTETLLLEW | SEQ ID NO: 60 |
| Var9-3Ec | CEEQQPWRAYLELLFPTETLLLEW | SEQ ID NO: 61 |
| Var15-2N | CDDDDDNPNYWARYANWLFTTPLLLLNGALLVEAEET | SEQ ID NO: 62 |
| Var16-2P | CDDDDDNPNYWARYAPWLFTTPLLLLPGALLVEAEE | SEQ ID NO: 63 |
| Var3-DQD | ADQDNPWRAYLDLLFPTDTLLLDLLWCG | SEQ ID NO: 218 |
| Var3-DDQ | ADDQNPWRAYLDLLFPTDTLLLDLLWC | SEQ ID NO: 219 |

TABLE 2
Non-limiting examples of pHLIP® sequences.

| Name | Sequence | SEQ ID NO: |
|---|---|---|
| Var14- | Ac-TEDADVLLALDLLLLPTTFLW | SEQ. ID NO. 64 |
| Rev | DAYRAWYPNQECA-Am | |
| Sh | AEQNPIYWARYADWLFTTPL | SEQ. ID NO. 65 |
| Sh-Cys | AEQNPIYWARYADWLFTTPCL | SEQ. ID NO. 66 |
| Cys-Sh | ACEQNPIYWARYADWLFTTPL | SEQ. ID NO. 67 |
| Sh-1Trp | AEQNPIYFARYADWLFTTPL | SEQ. ID NO. 68 |
| Sh-W2 | AEQNPIYFARYADLLFPTTLAW | SEQ ID NO. 69 |
| Sh-W1 | AEQNPIYWARYADLLFPTTLAF | SEQ ID NO. 70 |
| Sh-2W | AEQNPIYWARYADLLFPTTLAW | SEQ ID NO. 71 |
| Sh-1D | KEDQNPWARYADLLFPTTLAW | SEQ. ID NO. 72 |
| Sh-1Db | KEDQNPWARYADLLFPTTLW | SEQ ID NO. 73 |
| Var12-1D | ACEDQNPWARYADLLFPTTLAW | SEQ. ID NO. 74 |
| Var10-2D | ACEDQNPWARYADWLFPTTLLLLD | SEQ. ID NO. 75 |
| Var13-1E | ACEEQNPWARYAELLFPTTLAW | SEQ. ID NO. 76 |
| Var11-2E | ACEEQNPWARYAEWLFPTTLLLLE | SEQ. ID NO. 77 |
| Var7-3E | ACEEQNPWARYLEWLFPTETLLLEL | SEQ. ID NO. 78 |
| Var7-3Eb | ACEEQNPQAEYAEWLFPTTLLLLE | SEQ ID NO: 79 |

* Ac means Acetylated N-terminus; and Am means Amidated C-terminus

TABLE 3
Coded and exemplary non-coded amino acids including
L-isomers, D-isomers, alpha-isomers, beta-isomers,
glycol-, and methyl- modifications.

| No. | Abbrev | Name |
|---|---|---|
| 1 | Ala | Alanine |
| 2 | Arg | Arginine |
| 3 | Asn | Asparagine |
| 4 | Asp | Aspartic acid |
| 5 | Cys | Cysteine |
| 6 | Gln | Glutamine |
| 7 | Glu | Glutamic acid |
| 8 | Gly | Glycine |
| 9 | His | Histidine |
| 10 | Ile | Isoleucine |
| 11 | Leu | Leucine |
| 12 | Lys | Lysine |
| 13 | Met | Methionine |
| 14 | Phe | Phenylalanine |
| 15 | Pro | Proline |
| 16 | Ser | Serine |
| 17 | Thr | Threonine |
| 18 | Trp | Tryptophan |
| 19 | Tyr | Tyrosine |
| 20 | Val | Valine |
| 21 | Sec | Selenocysteine |
| 22 | Sem | Selenomethionine |
| 23 | Pyl | Pyrrolysine |
| 24 | Aad | Alpha-aminoadipic acid |
| 25 | Acpa | Amino-caprylic acid |
| 26 | Aecys | Aminoethyl cysteine |
| 27 | Afa | Aminophenyl acetate |
| 28 | Gaba | Gamma-aminobutyric acid |
| 29 | Aiba | Aminoisobutyric acid |
| 30 | Aile | Alloisoleucine |
| 31 | AIg | Allylglycine |
| 32 | Aba | Amino-butyric acid |
| 33 | Aphe | Amino-phenylalanine |
| 34 | Brphe | Bromo-phenylalanine |
| 35 | Cha | Cyclo-hexylalanine |
| 36 | Cit | Citrulline |
| 37 | Clala | Chloroalanine |
| 38 | Cie | Cycloleucine |
| 39 | Clphe | Fenclonine (or chlorophenylalanine) |
| 40 | Cya | Cysteic acid |
| 41 | Dab | Diaminobutyric acid |
| 42 | Dap | Diaminopropionic acid |
| 43 | Dap | Diaminopimelic acid |
| 44 | Dhp | Dehydro-proline |
| 45 | Dhphe | DOPA (or 3,4-dihydroxyphenylalanine) |
| 46 | Fphe | Fluorophenylalanine |
| 47 | Gaa | Glucosaminic acid |
| 48 | Gla | Gamma-carboxyglutamic acid |
| 49 | Hag | Homoarginine |
| 50 | Hlys | Hydroxylysine |
| 51 | Hnvl | Hydroxynorvaline |
| 52 | Hog | Homoglutamine |
| 53 | Hoph | Homophenylalanine |
| 54 | Has | Homoserine |
| 55 | Hse | Homocysteine |
| 56 | Hpr | Hydroxyproline |
| 57 | Iphe | Iodo-phenylalanine |
| 58 | Ise | Isoserine |
| 59 | Mle | Methyl-leucine |
| 60 | Msmet | Methionine-methylsulfonium chloride |
| 61 | Nala | Naphthyl-alanine |
| 62 | Nle | Norleucine (or 2-aminohexanoic acid) |
| 63 | Nmala | N-methyl-alanine |
| 64 | Nva | Norvaline (or 2-aminopentanoic acid) |
| 65 | Obser | O-benzyl-serine |
| 66 | Obtyr | O-benzyl-tyrosine |
| 67 | Oetyr | O-ethyl-tyrosine |
| 68 | Omser | O-methyl-serine |
| 69 | Omthr | O-methy-threonine |
| 70 | Omtyr | O-methyl-tyrosine |
| 71 | Orn | Ornithine |
| 72 | Pen | Penicillamine |
| 73 | Pga | Pyroglutamic acid |
| 74 | Pip | Pipecolic acid |
| 75 | Sar | Sarcosine |
| 76 | Tfa | Trifluoro-alanine |
| 77 | Thphe | Hydroxy-Dopa |
| 78 | Vig | Vinylglycine |
| 79 | Aaspa | Amino-aminoethylsulfanylpropanoic acid |
| 80 | Ahdna | Amino-hydroxy-dioxanonanolic acid |
| 81 | Ahoha | Amino-hydroxy-oxahexanoic acid |
| 82 | Ahsopa | Amino-hydroxyethylsulfanylpropanoic acid |
| 83 | Tyr(Me) | |
| 84 | MTrp | Methoxyphenyl-methylpropanyl oxycarbonylamino |
| 85 | pTyr | propanoic acid |
| 86 | pSer | Methyl-tryptophan |
| 87 | pThr | Phosphorylated Tyr |
| 88 | BLys | Phosphorylated Ser |
| 89 | Hyp | Phosphorylated Thr |
| 90 | Phg | BiotinLys |
| 91 | Cha | Hydroproline |
| 92 | Chg | Phenylglycine |
| 93 | Nal | Cyclohexyl-alanine |
| 94 | Pal | Cyclohexylglycine |
| 95 | Pra | Naphthylalanine |
| 96 | Gly(allyl) | Pyridyl-alanine |
| 97 | Pen | Propargylglycine |
| 98 | MetO | Pentenoic acid |
| 99 | Pca | Penicillamine |
| 100 | Ac-Lys | Methionine sulfoxide |
| | | Pyroglutamic acid |
| | | Acetylation of Lys |

TABLE 4
Non-limiting examples of protonatable residues
and their substitutions including L-isomers,
D- isomers, alpha-isomers, and beta-isomers.

| Original | |
|---|---|
| Residue | Exemplary amino acids substitution |
| Asp (D) | Glu (E); Gla (Gla); Aad (Aad) |
| Glu (E) | Asp (D); Gla (Gla); Aad (Aad) |

TABLE 5
Examples of coded amino acid substitutions.

| Original | |
|---|---|
| Residue | Substitution |
| Ala (A) | Gly; Ile; Leu; Met; Phe; Pro; Trp; Tyr; Val |
| Arg (R) | Lys |
| Asn (N) | Gln; His |
| Asp (D) | Glu |
| Cys (C) | Ser; Met |
| Gln (Q) | Asn; His |
| Glu (E) | Asp |
| Gly (G) | Ala; Ile; Leu; Met; Phe; Pro; Trp; Tyr; Val |
| His (H) | Asn; Gln |
| Ile (I) | Ala; Gly; Leu; Met; Phe; Pro; Trp; Tyr; Val |
| Leu (L) | Ala; Gly; Ile; Met; Phe; Pro; Trp; Tyr; Val |
| Lys (K) | Arg |
| Met (M) | Ala; Gly; Leu; Ile; Phe; Pro; Trp; Tyr; Val |
| Phe (F) | Ala; Gly; Leu; Ile; Met; Pro; Trp; Tyr; Val |
| Pro (P) | Ala; Gly; Leu; Ile; Met; Phe; Trp; Tyr; Val |
| Ser (S) | Thr |
| Thr (T) | Ser |
| Trp (W) | Ala; Gly; Leu; Ile; Met; Pro; Phe; Tyr; Val |
| Tyr (Y) | Ala; Gly; Leu; Ile; Met; Pro; Phe; Trp; Val |
| Val (V) | Ala; Gly; Leu; Ile; Met; Pro; Phe; Trp; Tyr |

TABLE 6
Non-limiting examples of membrane-inserting
sequences belonging to different groups of
pHLIP® peptides.

| Groups | Sequences (SEQ ID NO:) |
|---|---|
| WT-BRC | WARYADWLFTTPLLLLDLALL (SEQ ID NO: 80) |
| | YARYADWLFTTPLLLLDLALL (SEQ ID NO: 81) |
| | WARYSDWLFTTPLLLYDLGLL (SEQ ID NO: 82) |
| | WARYTDWFTTPLLLYDLALLA (SEQ ID NO: 83) |
| | WARYTDWLFTTPLLLYDLGLL (SEQ ID NO: 84) |
| | WARYADWLFTTPLLLLDLSLL (SEQ ID NO: 85) |
| WT-BRC | LLALDLLLLPTTFLWDAYRAW (SEQ ID NO: 86) |
| Reverse | LLALDLLLLPTTFLWDAYRAY(SEQ ID NO: 87) |
| | LLGLDYLLLPTTFLWDSYRAW (SEQ ID NO: 88) |
| | ALLALDYLLLPTTFWDTYRAW (SEQ ID NO: 89) |
| | LLGLDYLLLPTTFLWDTYRAW (SEQ ID NO: 90) |
| | LLSLDLLLLPTTFLWDAYRAW (SEQ ID NO: 91) |
| ATRAM | GLAGLLGLEGLLGLPLGLLEGLWLGL (SEQ ID NO: |
| | 92) |
| ATRAM | LGLWLGELLGLPLGLLGELGLLGALG (SEQ ID NO: |
| Reverse | 93) |
| Var3 | WRAYLDLLFPTDTLLLDLLW (SEQ ID NO: 94) |
| Var3 | WLLDLLLTDTPFLLDLYARW (SEQ ID NO: 95) |
| Reverse | |
| Var7 | WARYLEWLFPTETLLLEL (SEQ ID NO: 96) |
| | WAQYLELLFPTETLLLEW (SEQ ID NO: 97) |
| Var7 | LELLLTETPFLWELYRAW (SEQ ID NO: 98) |
| Reverse | WELLLTETPFLLELYQAW (SEQ ID NO: 99) |
| Single | WLFTTPLLLLNGALLVE (SEQ ID NO: 100) |
| D/E | WLFTTPLLLLPGALLVE (SEQ ID NO: 101) |
| | WARYADLLFPTTLAW (SEQ ID NO: 102) |
| Single | EVLLAGNLLLLPTTFLW (SEQ ID NO: 103) |
| D/E | EVLLAGPLLLLPTTFLW (SEQ ID NO: 104) |
| Reverse | WALTTPFLLDAYRAW (SEQ ID NO: 105) |
| pHLIP®- | NLEGFFATLGGEIALWSLVVLAIE (SEQ ID NO: |
| Rho | 106) |
| | EGFFATLGGEIALWSDVVLAIE (SEQ ID NO: 107) |
| | EGFFATLGGEIPLWSDVVLAIE (SEQ ID NO: 108) |
| pHLIP®- | EIALVVLSWLAIEGGLTAFFGELN (SEQ ID NO: |
| Rho | 109) |
| Reverse | EIALVVDSWLAIEGGLTAFFGE (SEQ ID NO: 110) |
| | EIALVVDSWLPIEGGLTAFFGE (SEQ ID NO: 111) |
| pHLIP®- | ILDLVFGLLFAVTSVDFLVQW (SEQ ID NO: 112) |
| CA9 | |
| pHLIP®- | WQVLFDVSTVAFLLGFVLDLI (SEQ ID NO: 113) |
| CA9 | |
| Reverse | |

Each protonatable residue (shown in bold) could be replaced by its substitution from Table 4. Each non-polar residue could be replaced by its coded amino acid substitution from Table 5, and/or non-coded amino acid substitutions from Table 3.

TABLE 7
Non-limiting examples of pHLIP® sequences.
A cysteine, a lysine, an azido-modified amino acid, or an alkynyl modified amino
acid can be incorporated at the N-terminal (first six residues) or C-terminal (last six
residues) parts of the peptides for conjugation with a cargo, and a linker.

| SEQ ID NO: | Name | Sequence |
|---|---|---|
| SEQ ID NO: 114 | WT-2D | AEQNPIYWARYADWLFTTPLLLLDLALLVDADET |
| SEQ ID NO: 115 | WT-6E | AEQNPIYWARYAEWLFTTPLLLLELALLVEAEET |
| SEQ ID NO: 116 | WT-3D | ADDQNPWRAYLDLLFPDTTDLLLLDLLWDADET |
| SEQ ID NO: 117 | WT-9E | AEEQNPWRAYLELLFPETTELLLLELLWEAEET |
| SEQ ID NO: 118 | WT-GlaD | AEQNPIYWARYAGlaWLFTTPLLLLDLALLVDADET |
| SEQ ID NO: 119 | WT-DGla | AEQNPIYWARYADWLFTTPLLLLGlaLALLVDADET |
| SEQ ID NO: 120 | WT-2Gla | AEQNPIYWARYAGlaWLFTTPLLLLGlaLALLVDADET |
| SEQ ID NO: 121 | WT-AadD | AEQNPIYWARYAAadWLFTTPLLLLDLALLVDADET |
| SEQ ID NO: 122 | WT-DAad | AEQNPIYWARYADWLFTTPLLLLAadLALLVDADET |
| SEQ ID NO: 123 | WT-2Aad | AEQNPIYWARYAAadWLFTTPLLLLAadLALLVDADET |
| SEQ ID NO: 124 | WT-GlaAad | AEQNPIYWARYAGlaWLFTTPLLLLAadLALLVDADET |
| SEQ ID NO: 125 | WT-AadGla | AEQNPIYWARYAAadWLFTTPLLLLGlaLALLVDADET |
| SEQ ID NO: 126 | WT-1 | GGEQNPIYWARYADWLFTTPLLLLDLALLVDADEGT |
| SEQ ID NO: 127 | WT-2 | GGEQNPIYWARYADWLFTTPLLLLDLALLVDADEGT |
| SEQ ID NO: 128 | WT-3 | AAEQNPIYWARYADWLFTTPLLLLDLALLVDADEGT |
| SEQ ID NO: 129 | WT-4 | AEQNPIYWARYADWLFTTPLLLLDLALLVDADEGT |
| SEQ ID NO: 130 | WT-2N | AEQNPIYWARYANWLFTTPLLLLNLALLVDADEGT |
| SEQ ID NO: 131 | WT-2K | AEQNPIYWARYAKWLFTTPLLLLKLALLVDADEGT |
| SEQ ID NO: 132 | WT-2DNANQ | GGEQNPIYWARYADWLFTTPLLLLDLALLVNANQGT |
| SEQ ID NO: 133 | WT-D25A | AAEQNPIYWARYADWLFTTPLLLLALALLVDADEGT |
| SEQ ID NO: 134 | WT-D14A | AAEQNPIYWARYAAWLFTTPLLLLDLALLVDADEGT |
| SEQ ID NO: 135 | WT-P20A | AAEQNPIYWARYADWLFTTALLLLDLALLVDADEGT |
| SEQ ID NO: 136 | WT-D25E | AAEQNPIYWARYADWLFTTPLLLLELALLVDADEGT |
| SEQ ID NO: 137 | WT-D14E | AAEQNPIYWARYAEWLFTTPLLLLDLALLVDADEGT |
| SEQ ID NO: 138 | WT-3D-2 | AAEQNPIIYWARYADWLFTDLPLLLLDLLALLVDADEGT |
| SEQ ID NO: 139 | WT-R11Q | GEQNPIYWAQYADWLFTTPLLLLDLALLVDADEG |
| SEQ ID NO: 140 | WT-D25Up | GGEQNPIYWARYADWLFTTPLLLDLLALLVDADEG |
| SEQ ID NO: 141 | WT-D25Down | GGEQNPIYWARYADWLFTTPLLLLLDALLVDADEG |
| SEQ ID NO: 142 | WT-D14Up | GGEQNPIYWARYDAWLFTTPLLLLDLALLVDADEGT |
| SEQ ID NO: 143 | WT-D14Down | GGEQNPIYWARYAWDLFTTPLLLLDLALLVDADEG |
| SEQ ID NO: 144 | WT-P20G | AAEQNPIYWARYADWLFTTGLLLLDLALLVDADEGT |
| SEQ ID NO: 145 | WT-DH | DDDEDNPIYWARYADWLFTTPLLLLHGALLVDAD |
| SEQ ID NO: 146 | WT-2H | DDDEDNPIYWARYAHWLFTTPLLLLHGALLVDADE |
| SEQ ID NO: 147 | WT-L16H | CEQNPIYWARYADWHFTTPLLLLDLALLVDADE |
| SEQ ID NO: 148 | WT-1Wa | AEQNPIYWARYADFLFTTPLLLLDLALLVDADET |
| SEQ ID NO: 149 | WT-1Wb | AEQNPIYFARYADWLFTTPLLLLDLALLVDADE |
| SEQ ID NO: 150 | WT-1Wc | AEQNPIYFARYADFLFTTPLLLLDLALLWDADET |
| SEQ ID NO: 151 | WT-W6 | ADNNPWIYARYADLTTFPLLLLDLALLVDFDD |
| SEQ ID NO: 152 | WT-W17 | ADNNPFIYARYADLTTWPLLLLDLALLVDFDD |
| SEQ ID NO: 153 | WT-W30 | ADNNPFIYARYADLTTFPLLLLDLALLVDWDD |
| SEQ ID NO: 154 | WT-W17-P7 | ADNNPFPYARYADLTTWILLLLDLALLVDFDD |
| SEQ ID NO: 155 | WT-W39-R11 | ADNNPFIYAYRADLTTFPLLLLDLALLVDWDD |
| SEQ ID NO: 156 | WT-W30-R15 | ADNNPFIYATYADLRTFPLLLLDLALLVDWDD |
| SEQ ID NO: 157 | WT-Rev | Ac-TEDADVLLALDLLLLPTTFLWDAYRAWYPNQEA-Am |
| SEQ ID NO: 158 | Var1-3D | AEDQNPYWARYADWLFTTPLLLLDLALLVD |
| SEQ ID NO: 159 | Var1-1D2E | AEDQNPYWARYADWLFTTPLLLLELALLVE |
| SEQ ID NO: 160 | Var2-3D | AEDQNPYWRAYADLFTPLTLLDLLALWD |
| SEQ ID NO: 161 | Var3-3D | ADDQNPWRAYLDLLFPTDTLLLDLLW |
| SEQ ID NO: 162 | Var3-WT | ADDQNPWRAYLDLLFPTDTLLLDLLWDADE |
| SEQ ID NO: 163 | Var3-Gla2D | ADDQNPWRAYLGlaLLFPTDTLLLDLLW |
| SEQ ID NO: 164 | Var3-DGlaD | ADDQNPWRAYLDLLFPTGlaTLLLDLLW |
| SEQ ID NO: 165 | Var3-2DGla | ADDQNPWRAYLDLLFPTDTLLLGlaLLW |
| SEQ ID NO: 166 | Var3-2GlaD | ADDQNPWRAYLGlaLLFPTGlaTLLLDLLW |
| SEQ ID NO: 167 | Var3-GlaDGla | ADDQNPWRAYLGlaLLFPTDTLLLGlaLLW |
| SEQ ID NO: 168 | Var3-D2Gla | ADDQNPWRAYLDLLFPTGlaTLLLGlaLLW |
| SEQ ID NO: 169 | Var3-3Gla | ADDQNPWRAYLGlaLLFPTGlaTLLLGlaLLW |
| SEQ ID NO: 170 | Var3-Aad2D | ADDQNPWRAYLAadLLFPTDTLLLDLLW |
| SEQ ID NO: 171 | Var3-DAadD | ADDQNPWRAYLDLLFPTAadTLLLDLLW |
| SEQ ID NO: 172 | Var3-2DAad | ADDQNPWRAYLDLLFPTDTLLLAadLLW |
| SEQ ID NO: 173 | Var3-2AadD | ADDQNPWRAYLAadLLFPTAadTLLLDLLW |
| SEQ ID NO: 174 | Var3-AadDAad | ADDQNPWRAYLAadLLFPTDTLLLAadLLW |
| SEQ ID NO: 175 | Var3-D2Aad | ADDQNPWRAYLDLLFPTAadTLLLAadLLW |
| SEQ ID NO: 176 | Var3-3 Aad | ADDQNPWRAYLAadLLFPTAadTLLLAadLLW |
| SEQ ID NO: 177 | Var3-GlaAadD | ADDQNPWRAYLGlaLLFPTAadTLLLDLLW |
| SEQ ID NO: 178 | Var3-GlaDAad | ADDQNPWRAYLGlaLLFPTDTLLLAadLLW |
| SEQ ID NO: 179 | Var3-2GlaAad | ADDQNPWRAYLGlaLLFPTGlaTLLLAadLLW |
| SEQ ID NO: 180 | Var3-AadGlaD | ADDQNPWRAYLAadLLFPTGlaTLLLDLLW |
| SEQ ID NO: 181 | Var3-AadDGla | ADDQNPWRAYLAadLLFPTDTLLLGlaLLW |
| SEQ ID NO: 182 | Var3-GlaAadGla | ADDQNPWRAYLGlaLLFPTAadTLLLGlaLLW |
| SEQ ID NO: 183 | Var3-GLL | GEEQNPWLGAYLDLLFPLELLGLLELGLW |
| SEQ ID NO: 184 | Var3-M | ADDDDDDPWQAYLDLLFPTDTLLLDLLW |
| SEQ ID NO: 185 | Var4-3E | AEEQNPWRAYLELLFPTETLLLELLW |
| SEQ ID NO: 186 | Var5-3Da | ADDQNPWARYLDWLFPTDTLLLDL |
| SEQ ID NO: 187 | Var6-3Db | DNNNPWRAYLDLLFPTDTLLLDW |
| SEQ ID NO: 188 | Var7-3E | AEEQNPWARYLEWLFPTETLLLEL |
| SEQ ID NO: 189 | Var7-M | DDDDDDPWQAYLDLFPTDTLALDLW |
| SEQ ID NO: 190 | Var8-3E | EEQQPWAQYLELLFPTETLLLEW |
| SEQ ID NO: 191 | Var9-3E | EEQQPWRAYLELLFPTETLLLEW |
| SEQ ID NO: 192 | Var10-2D | AEDQNPWARYADWLFPTTLLLLD |
| SEQ ID NO: 193 | Var11-2E | AEEQNPWARYAEWLFPTTLLLLE |
| SEQ ID NO: 194 | Var12-1D | AEDQNPWARYADLLFPTTLAW |
| SEQ ID NO: 195 | Var13-1E | AEEQNPWARYAELLFPTTLAW |
| SEQ ID NO: 196 | Var15-2N | DDDDDNPNYWARYANWLFTTPLLLLNGALLVEAEET |
| SEQ ID NO: 197 | Var16-2P | DDDDDNPNYWARYAPWLFTTPLLLLPGALLVEAEET |
| SEQ ID NO: 198 | Var17 | AEQNPIYFARYADFLFTTPLLLLDLALLWDADET |
| SEQ ID NO: 199 | Var18 | AEQNPIYWARYADFLFTTPLLLLDLALLVDADET |
| SEQ ID NO: 200 | Var19a | AEQNPIYWARYADWLFTTPL |
| SEQ ID NO: 201 | Var20 | AEQNPIYFARYADLLFPTTLAW |
| SEQ ID NO: 202 | Var21 | AEQNPIYWARYADLLFPTTLAF |
| SEQ ID NO: 203 | Var22 | AEQNPIYWARYADLLFPTTLAW |
| SEQ ID NO: 204 | Var23 | AEQNPIYFARYADWLFTTPL |
| SEQ ID NO: 205 | Var24 | EDQNPWARYADLLFPTTLAW |
| SEQ ID NO: 206 | ATRAM | GLAGLAGLLGLEGLLGLPLGLLEGLWLGLELEGN |
| SEQ ID NO: 207 | pHLIP®-CA9 | EQNPIYILDLVFGLLFAVTSVDFLVQWDDAGD |
| SEQ ID NO: 208 | pHLIP®-Rho | NLEGFFATLGGEIALWSLVVLAIE |
| SEQ ID NO: 209 | pHLIP®-RhoM1 | NNEGFFATLGGEIALWSDVVLAIE |
| SEQ ID NO: 210 | pHLIP®-RhoM2 | DNNEGFFATLGGEIPLWSDVVLAIE |

In some aspects, the pHLIP® peptide comprises a sequence selected from the group consisting of any one of SEQ ID NOs. 1-219. In some aspects, the pHLIP® peptide of the group consisting of any one of SEQ ID NOs. 1-219. In other aspects, the pHLIP® peptide has a sequence comprising the sequence ADQDNPWRAYLDLLFPTDTLLLDLLWCG (SEQ ID NO: 218). In some aspects, the pHLIP® peptide has a sequence comprising the sequence ADDQNPWRAYLDLLFPTDTLLLDLLWCG (SEQ ID NO: 219). In some aspects, the pHILIP® peptide has a sequence consisting of the sequence ADQDNPWRAYLDLLFPTDTLLLDLLWCG (SEQ ID NO: 218). In some aspects, the pHLIP® peptide has a sequence consisting of the sequence

(SEQ ID NO: 219)
ADDQNPWRAYLDLLFPTDTLLLDLLWCG.

### Linker

A Linker can be relatively small, e.g., only a few atoms, to a rather large polymer of 4-5 kDa. A Linker can react on one end with a free thiol to spontaneously form a disulfide bond, with thiopyridine as a leaving group, and on the other end reacts with activated with amine or hydroxyl groups in the presence of DIPEA, and in some cases DMAP or other activator base, to form a carbamate or carbonate, respectively. This material can be used if pHLIP® (A) is protected at its amino terminus, such as with N-acetylation. This material can also be reacted with pHLIP® bearing a cysteine residue or with a thiol-bearing linker for subsequent conjugation to pHLIP®, and forms a conjugate by disulfide exchange with thiopyridine as a leaving group. This material can be used to form a conjugate with pHLIP® bearing a lysine residue, if pHLIP® is protected at its amino terminus, such as with N-acetylation.

In some aspects, a succinimidyl 3-(2-pyridyldithio) propionate (SPDP) cross-linker is used. SPDP is a short-chain crosslinker for amine-to-sulfhydryl conjugation via NHS-ester and pyridyldithiol reactive groups that form cleavable (reducible) disulfide bonds with cysteine sulfhydryls. SPDP is used to activate NH₂ derivative of cCDN (of e.g., c[3′-AHC-G(2′,5′)pA(3′,5′)p], or c[G(2′,5′)p-2′-AHC-A(3′,5′)p]), purify and exchange disulfide with SH of single cysteine (Cys) at the C-terminus of pHLIP® peptide to obtain pHLIP®-S-S-cCDN.

In some aspects, the following cross-linkers can be used: LC-SPDP (succinimidyl 6-(3 (2-pyridyldithio) propionamido) hexanoate); Sulfo-LC-SPDP (sulfosuccinimidyl 6-(3′-(2-pyridyldithio) propionamido) hexanoate); PEG4-SPDP (PEGylated, long-chain SPDP crosslinker); PEG12-SPDP (PEGylated, long-chain SPDP crosslinker); SMCC (succinimidyl 4-(N-maleimidomethyl)cyclohexane-1-carboxylate); Sulfo-SMCC (sulfosuccinimidyl 4-(N-maleimidomethyl)cyclohexane-1-carboxylate); SMPT (4-succinimidyloxycarbonyl-alpha-methyl-α(2-pyridyldithio) toluene); DTME (dithiobismaleimidoethane).

In some aspects, the Linker is attached to the C-terminus of the pHLIP® peptide.

### Compositions

In one aspect, the compositions disclosed herein comprise the following structure:

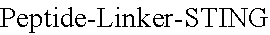

wherein Peptide is a pHILIP® peptide,
wherein Linker is a cleavable linker;
   wherein “STING” is a STING agonist selected from the group consisting of diABZI, ADU-S100 (MIW815), MK-1454, MK-2118, SB11285, GSK3745417, BMS-986301, BI1387446, E7766, TAK-676, SNZ281, IMSA-101, IMSA-201, SYNB1891, JNJ-4412, JNJ-6196, BI-STING, GSK532, TTI-10001, CRD5500, LB-061, ALG-031048, RVU312-4787, TAK-500, CL656, CDK-002, CF501, CF502, CF503, CF504, CF505, CF506, CF507, CF508, CF509, CF510, CF511, and derivatives thereof; and wherein each “-” is a covalent bond

In some aspects, the pHLIP® peptide has the following sequence: XₙYₘ; YₘXₙ; XₙYₘXⱼ; YₘXₙYᵢ; YₘXₙYᵢXⱼ; XₙYₘXⱼYᵢ; YₘXₙYᵢXᵢYₗ; XₙYₘXⱼYᵢXₗ; YₘXₙYᵢXⱼYₗXₕ; XₙYₘXⱼYᵢXₕY_{g}; YₘXₙYᵢXⱼYₗXₕ Y_{g}; XₙYₘXⱼYᵢXₕY_{g}X_{f}; (XY)ₙ; (YX)ₙ; (XY)ₙ Yₘ; (YX)ₙYₘ; (XY)ₙXₘ; (YX)ₙXₘ; Yₘ (XY)ₙ; Yₘ(YX)ₙ; Xₙ(XY)ₘ; Xₙ(YX)ₘ; (XY)ₙYₘ (XY)ᵢ; (YX)ₙYₘ (YX)ᵢ; (XY)ₙXₘ(XY)ᵢ; (YX)ₙXₘ(YX)ᵢ; Yₘ (XY)ₙ; Yₘ (YX)ₙ; Xₙ(XY)ₘ; or Xₙ(YX)ₘ; wherein,
(i) each Y is, independently, a non-polar amino acid with solvation energy, DG_{X}^{cor}>+0.50, or Glycine (Gly);
(ii) each X is, independently, a protonatable amino acid,
(iii) n, m, i, j, 1, h, g, f are each, independently, an integer from 1 to 8.

In some aspects, the pHLIP® peptide comprises a sequence comprising the sequence ADDQNPWRAYLDLLFPTDTLLLDLLWXG (SEQ ID NO: 220), wherein “X” is a functional group for conjugation purposes, selected from lysine (Lys), cysteine (Cys), Azido-containing amino acid or other modified amino acids. In some aspects, the pHLIP® peptide comprises a sequence consisting of the sequence ADDQNPWRAYLDLLFPTDTLLLDLLWXG (SEQ ID NO: 220), wherein “X” is a functional group for conjugation purposes, selected from lysine (Lys), cysteine (Cys), Azido-containing amino acid or other modified amino acids.

In some aspects, the pHLIP® peptide comprises a sequence selected from the group consisting of any one of SEQ ID NOs. 1-219. In some aspects, the pHLIP® peptide has a sequence selected from of the group consisting of any one of SEQ ID NOs. 1-219. In other aspects, the pHLIP® peptide has a sequence comprising the sequence ADQDNPWRAYLDLLFPTDTLLLDLLWCG (SEQ ID NO: 218). In some aspects, the pHLIP® peptide of the pHLIP® peptide has a sequence comprising the sequence ADDQNPWRAYLDLLFPTDTLLLDLLWCG (SEQ ID NO: 219). In some aspects, the pHLIP® peptide has a sequence consisting of the sequence ADQDNPWRAYLDLLFPTDTLLLDLLWCG (SEQ ID NO: 218). In some aspects, the pHLIP® peptide has a sequence consisting of the sequence

(SEQ ID NO: 219)
ADDQNPWRAYLDLLFPTDTLLLDLLWCG.

In some aspects, the STING agonist is diABZI. In other aspects, the STING agonist is MK-1454. In still yet another aspect, the STING agonist is MK-2118. In another aspect, the STING agonist is TAK-676. In other aspects, the STING agonist is BMS-986301. In yet another aspect, the STING agonist is BI1387446.

In some aspects, the compositions disclosed herein comprise the following structure:

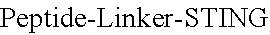

wherein Peptide is a pHLIP® peptide comprising the sequence

(SEQ ID NO: 218)
ADQDNPWRAYLDLLFPTDTLLLDLLWCG
or
(SEQ ID NO: 219)
ADDQNPWRAYLDLLFPTDTLLLDLLWCG,

wherein Linker is a cleavable linker;
wherein STING is a STING agonist; and
wherein each “-” is a covalent bond are also disclosed herein.

In some aspects, the pHLIP® peptide comprises the sequence ADQDNPWRAYLDLLFPTDTLLLDLLWCG (SEQ ID NO: 218). In some aspects, the pHLIP® peptide comprises the sequence ADDQNPWRAYLDLLFPTDTLLLDLLWCG (SEQ ID NO: 219). In some aspects, the pHLIP® peptide consists of the sequence

(SEQ ID NO: 218)
ADQDNPWRAYLDLLFPTDTLLLDLLWCG
or
(SEQ ID NO: 219)
ADDQNPWRAYLDLLFPTDTLLLDLLWCG.

In some aspects, the pHLIP® peptide consists of the sequence ADQDNPWRAYLDLLFPTDTLLLDLLWCG (SEQ ID NO: 218). In some aspects, the pHLIP® peptide consists of the sequence ADDQNPWRAYLDLLFPTDTLLLDLLWCG (SEQ ID NO: 219).

In some aspects, the STING agonist comprises a cyclic dinucleotide (CDN). In some aspects, the STING agonist comprises a cyclic purine dinucleotide. In some aspects, the STING agonist is a non-nucleotide small molecule. In some aspects, the STING agonist is selected from the group consisting of diABZI, ADU-S100 (MIW815), MK-1454, MK-2118, SB11285, GSK3745417, BMS-986301, BI1387446, E7766, TAK-676, SNZ281, IMSA-101, IMSA-201, SYNB1891, JNJ-4412, JNJ-6196, BI-STING, GSK532, TTI-10001, CRD5500, LB-061, ALG-031048, RVU312-4787, TAK-500, CL656, CDK-002, CF501, CF502, CF503, CF504, CF505, CF506, CF507, CF508, CF509, CF510, CF511, and derivatives thereof. In some aspects, the STING agonist is diABZI. In some aspects, the STING agonist is MK-1454. In some aspects, the STING agonist is MK-2118. In some aspects, the STING agonist is TAK-676. In other aspects, the STING agonist is BMS-986301. In some aspects, the STING agonist is BI1387446.

In some aspects, all amino acids in the pHLIP® peptide of the compositions disclosed herein are D-amino acids.

In some aspects, the Linker according to the present disclosure comprise a disulfide bond or an acid-labile bond. In some aspects, the Linker is self-immolative. In some aspects, the composition has the following structure:

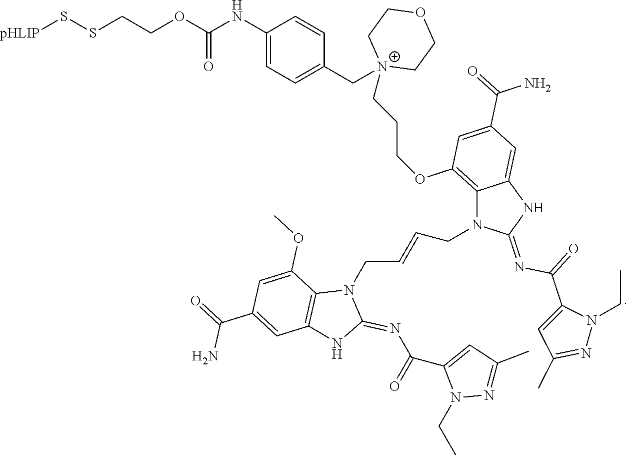

### Methods of Treatment

Methods of using the compositions described herein for treating cancer in a subject in need thereof comprising administering a composition or pharmaceutical composition disclosed herein are also provided. In some aspects, the cancer is a solid tumor. In some aspects, the composition or pharmaceutical composition disclosed herein is injected directly into a tumor mass. In some aspects, the subject is a human.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### EXAMPLES

### Example 1: Synthesis of pHLIP®-STINGa

A diABZI STING agonist was modified with a linker to prepare o-pyridyl-dithioethyl-carbamoyl-PAB-STING (Pys-PAB-STINGa). The agent was synthesized and purified by Iris Biotech GmbH. All pHLIP peptides used in the study were synthesized and purified at CSBio. For conjugation with Pys-PAB-STINGa the following pHLIP sequences with single Cys residues at the membrane-inserting ends of the peptides were used:
pHLIP(Laa): ADDQNPWRAYLDLLFPTDTLLLDLLWCG (SEQ ID NO: 218) in which all amino acids are in the L-configuration and
pHLIP(Daa): ADQDNPWRAYLDLLFPTDTLLLDLLWCG (SEQ ID NO: 219) in which all amino acids are in the D-configuration.

pHLIP® peptides and Pys-PAB-STINGa were mixed in dimethyl sulfoxide (DMSO) at a molar ratio 1:1. Sodium phosphate buffer (100 mM) containing 150 mM NaCl at pH 7.4 saturated with argon was added to the reaction mix (1/10 of the total volume) and the reaction mixture was kept for 2 hours at room temperature (RT). pHLIP®-STINGa constructs were purified by reverse phase high-performance liquid chromatography (HPLC) using Zorbax SB-C18, 9.4×250 mm, 5 μm column (Agilent Technology) with a gradient from 10% to 75% acetonitrile in water containing 0.05% of trifluoroacetic acid (TFA). For preparation of fluorescent versions of the agents, A1647-pHLIP®-STINGa and ICG-pHLIP-STINGa, N-acetylated versions of the pHLIP peptide AKDDQNPWRAYLDLLFPTDTLLLDLLWCG (SEQ ID NO: 221) in which all amino acids are in the L-configuration was used. First, pHLIP® was conjugated with Pys-PAB-STING, followed by purification. Then, either ICG-NHS ester (Iris Biotech GmbH) or Alexa647-NHS ester (Life Technologies) was conjugated with the lysine residue at the N-terminal end of pHLIP in DMSO at molar ratio of 1:1.5. Sodium bicarbonate buffer (100 mM) at pH 8.3 was added to the reaction mix (1/10 of the total volume) and the reaction mixture was kept at RT until the conjugation was completed. The final purification was performed as described above. The products were lyophilized and characterized by matrix-assisted laser desorption/ionization-time of flight (MALDI-TOF) mass-spectrometry and analytical HPLC. The concentration of pHLIP-STINGa conjugates was determined by absorbance using the following molar extinction coefficients: for pHLIP-STINGa ε₃₂₂=46,200 M⁻¹ cm⁻¹, for ICG-pHLIP-STINGa ε₈₀₀=137,000 M⁻¹ cm⁻¹ and for A1647-pHLIP-STINGa ε₆₅₁=270,000 M⁻¹ cm⁻¹.

### Example 2: Stability in Mouse and Human Plasma

To establish the stability of pHLIP(Laa)-STINGa and pHLIP(Daa)-STINGa in plasma, pHLIP-STINGa was mixed with single donor human or BALB/c mouse plasma (Innovative Research) at a concentration of 200 μM, and kept in plasma for 0, 2, 4 or 24 hours at 37° C. Plasma proteins were precipitated by methanol (1:5 volume ratio of plasma to methanol) and centrifugated for 10 min at 13.4 rpm. The supernatant was collected and analyzed by HPLC using a Zorbax SB-C18 4.6×250 mm, 5 μm column with a gradient from 10% to 75% acetonitrile in water containing 0.05% TFA. Chromatograms were recorded at 220 nm, 280 nm and 320 nm. As controls pHLIP(Laa), pHLIP(Daa), Pys-PAB-STINGa, SH-PAB-STINGa, where Pys-PAB-STINGa was conjugated with a Cys residue, and diABZI (Invivogen) were analyzed for stability in plasma under the same conditions. The results are provided in Table 8 below.

TABLE 8

| Time, | pHLIP(Laa)-STINGa | | pHLIP(Daa)-STINGa | |
|---|---|---|---|---|
| h | mouse | human | mouse | human |
| 0 | 99.8 (0) | | 99.4 (0) | |
| 2 | 90.0 (5.5) | 95.1 (3.9) | 90.9 (51.7) | 99.2 (77.2) |
| 4 | 82.7 (16.3) | 91.4 (5.3) | 84.5 (59.6) | 97.6 (84.4) |
| 24 | 57.0 (33.1) | 64.5 (20.5) | 58.2 (75.9) | 89.6 (87.7) |

### Example 3: Self-Immolation Kinetics

To trigger self-immolation of the linker, a solution of pHLIP(Laa)-STINGa was treated with dithiothreitol (DTT). At different time points (from 30 min to 2 hours of treatment) the samples were analyzed by HPLC using a Zorbax SB-C18 4.6×250 mm, 5 μm column with a gradient from 10% to 75% acetonitrile in water containing 0.05% TFA. The chromatograms were recorded at 220 nm, 280 nm and 320 nm. diABZI and pHLIP were used as controls at the same HPLC conditions.

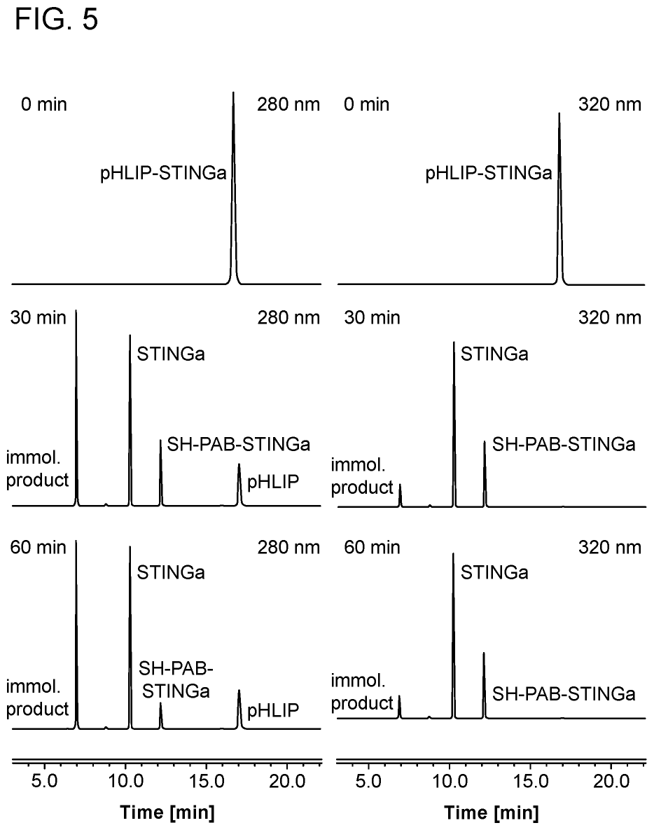

The results of the study are provided in FIG. 5.

### Example 4: Biophysics Studies

The interactions of pHLIP(Laa)-STINGa with liposomes were investigated by recording the construct's fluorescence and circular dichroism (CD) using a PC1 spectrofluorometer (ISS) and a MOS-450 spectrometer (Bio-Logic Science Instruments), respectively, with temperature control set to 25° C. Liposomes, constituting of large unilamellar vesicles were prepared by extrusion. 1-palmitoyl-2-oleoyl-sn-glycero-3-phosphocholine (POPC) lipids (Avanti Polar Lipids) in chloroform were desolvated on a rotary evaporator and dried under vacuum for a minimum of 2 hours. The phospholipid film was rehydrated in 2 mM citrate phosphate buffer, pH 7.3, vortexed, and passed through the extruder (using a 50 nm membrane pore size) 21 times.

Fluorescence spectra were recorded from 310 nm to 550 nm at an excitation wavelength of 295 nm and 1.0 mm sized slits. The excitation polarizer was set to 54.7 degrees (“magic angle”) while the emission polarizer was set to 0 degrees in order to reduce Wood's anomalies. CD spectra were recorded from 190 to 260 nm with step size of 1 nm. The concentrations of pHLIP-STINGa and POPC were 7 UM and 1.4 mM, respectively. Also, the fluorescence of STINGa (diABZI) was recorded when excited at the 295 nm and 350 nm wavelengths.

The pH-dependent insertion of pHLIP-STINGa into the lipid bilayer of POPC liposomes was studied by monitoring either the changes in fluorescence intensity at 400 nm or changes in the molar ellipticity at 230 nm as a function of pH. After the addition of aliquots of citric acid, the pHs of solutions containing pHLIP-STINGa and POPC liposomes were measured using an Orion PerHecT ROSS Combination pH Micro Electrode and an Orion Dual Star pH and ISE Benchtop Meter. The normalized fluorescence intensity or millidegree ellipticity values were plotted as a function of pH. The pH-dependence was fit with the Henderson-Hasselbach equation to determine the cooperativity (n) and the mid-point (pK) of transition. The fitting equations used were

$$pH dependence = SII + \frac{SIII - SII}{1 + {10}^{n ( pH - pK )}} for a single transition and$$

$$pH dependence = SII + \frac{{SII}^{′} - SII}{1 + {10}^{n 1 ( pH - pK 1 )}} + \frac{SIII - SII ′}{1 + {10}^{n 2 ( pH - pK 2 )}} for two transitions .$$

where SII and SIII represent spectral signals in state II and III, respectively, and SII′ represents the CD signal in intermediate between II and III state.

Fluorescence kinetics was measured using a SFM-300 mixing system (Bio-Logic Science Instruments) in combination with the MOS-450 spectrometer with temperature control set to 25° C. All samples were degassed before measurements to minimize air bubbles in the samples. pHLIP-STINGa and POPC samples were incubated overnight to reach equilibrium, when most of the agent was associated with liposome lipid bilayers. To follow pHLIP-STINGa insertion into a membrane, a solution containing 14 μM pHLIP-STINGa and 2.8 mM POPC was mixed with citric acid to lower the pH from pH 8 to 3.5. To monitor fluorescence intensity changes during pHLIP-STINGa insertion into POPC liposomes induced by the pH drop, the emission signal was observed through a cut-off 320 nm filter at an excitation of 295 nm.

For oriented circular dichroism (OCD) measurements, supported bilayers were prepared on quartz slides with special polish for far UV measurements (Starna). The procedure of slide cleaning included the following steps: 1) soaking in cuvette cleaner solution for 24 hours, 2) rinsing with de-ionized distilled water, 3) sonicating for 10 min in 2-propanol, 4) sonicating in acetone, 5) sonicating in 2-propanol once again, 6) rinsing with de-ionized water, 7) soaking in a piranha solution consisting of 25% hydrogen peroxide and 75% sulfuric acid, and 8) rinsing with Milli-Q purified water. A POPC lipid monolayer was deposited on a quartz substrate by the Langmuir-Blodgett (LB) method using (KSV minitrough). For the LB deposition, a small amount of POPC lipid in chloroform was spread on the surface of the subphase and solvent was allowed to evaporate for about 10 min. Next, the monolayer was compressed to 32 mN/m. When the surface pressure was stabilized the first slide was inserted into the trough and held there for 60 seconds so the surface pressure would stabilized again, then it was pulled out from the subphase with speed of 10 mm/min. The second layer was created by fusion with POPC vesicles. About 80 μl of either samples containing 7 μM pHLIP-STINGa and 0.7 mM POPC in 2 mM pH 5.0 or in 2 mM pH 3.3 citrate phosphate buffers or POPC blank containing 0.7 mM POPC (no pHLIP-STINGa) in citrate phosphate buffer was spread onto the slide. The process was repeated for eight more slides, then they were stacked on top of each other. The spacers between the slides kept them from sticking to each other. The “0-hour” OCD spectra were measured for samples at both pHs and POPC blank. Then, slides were kept at 100% humidity at 4° C. for 6 hours. After 6 hours, excess solution was shaken off each slide and replaced with 80 μL of buffer of corresponding pH. The slides were again stacked together while filling with the buffer to have a complete set of 8 slides (16 bilayers) and stored at 100% humidity at 4° C. for another 6 hours. At the end of the 12-hour incubation period, the “12-hour” OCD spectra were measured. The POPC blank OCD spectrum was subtracted from the OCD spectra of samples.

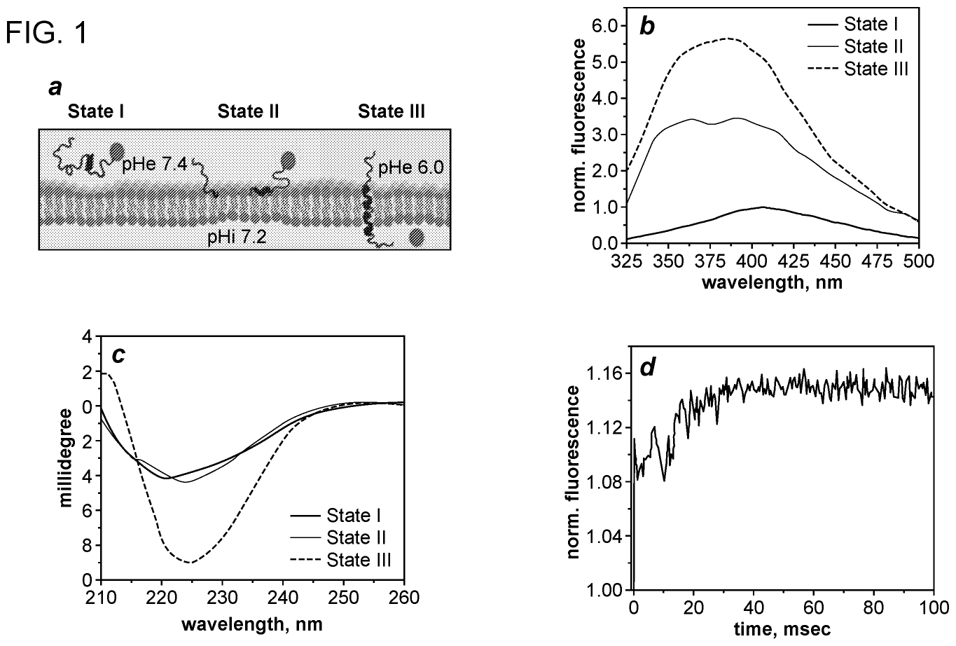

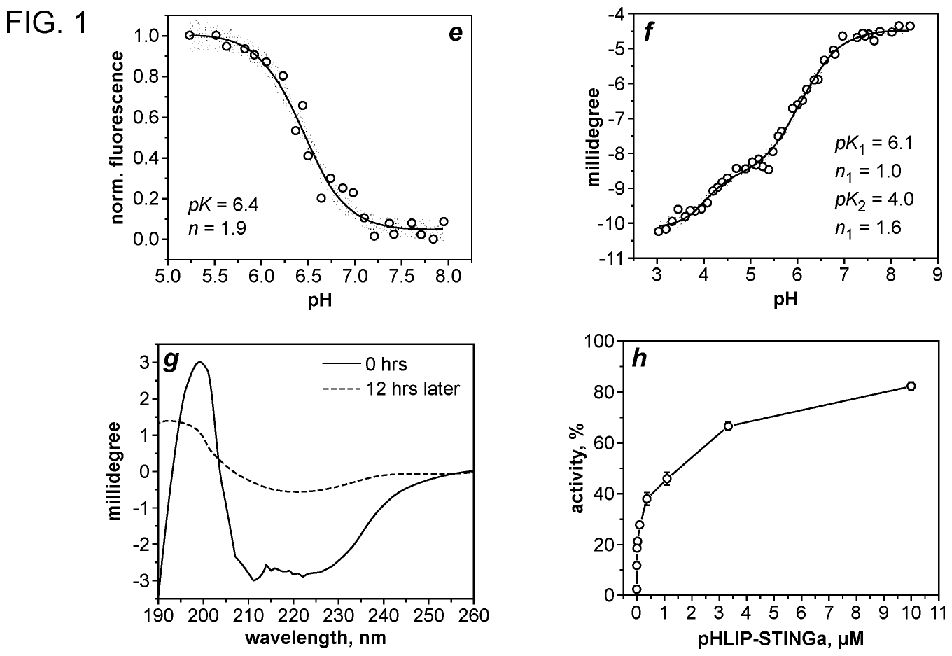

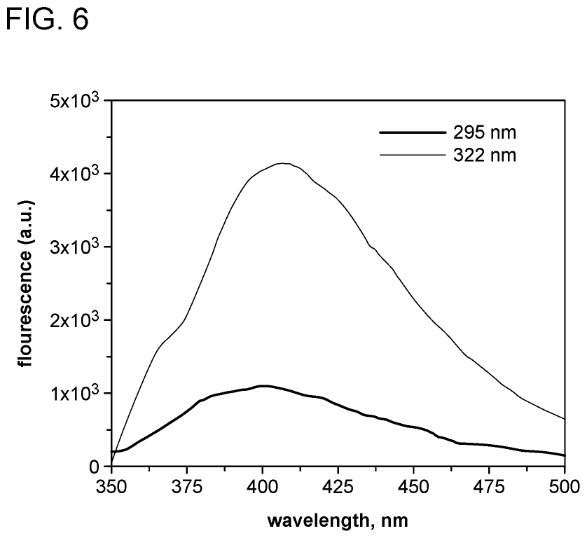

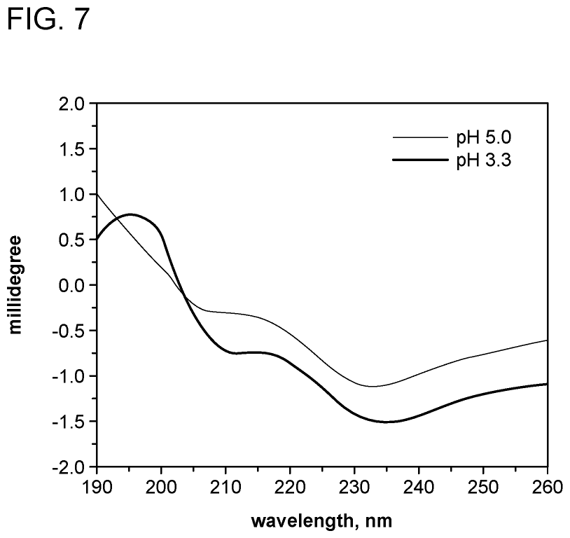

All data were fit to the appropriate equations by nonlinear least squares curve fitting procedures employing the Levenberg Marquardt algorithm using Origin 8.5. The characterization of the pHLIP-STINGa is provided in FIGS. 1, 5, and 6.

### Example 5: Activation of IFN in Cells

THP-1-Blue™-ISG cells (Invivogen) expressing an interferon (IFN) regulatory factor (IRF)-inducible secreted embryonic alkaline phosphatase (SEAP) reporter construct were used. Cells were maintained in RPMI growth medium supplemented with L-glutamine, sodium pyruvate, 10% fetal bovine serum (FBS), normocin and ciprofloxacin hydrochloride in a humidified atmosphere of 5% CO₂ and 95% air at 37° C. Cells were seeded in 96-well plates at a density of 75,000 cells/well. To generate M2 polarized macrophages, cells were treated first with 185 ng/ml phorbol 12-myristate 13-acetate (PMA) for 6 hours and then 20 ng/ml of interleukin 4 (IL-4) IL-4 and 20 ng/ml of interleukin 13 (IL-13) (both from PeproTech) were added for another 16 hours of treatment. At the completion of polarization, the growth medium was replaced with Dulbecco's Modified Eagle Medium (DMEM) medium without FBS, pH 6.9, containing increasing amounts of pHLIP(Laa)-STINGa or STINGa (up to 10.0 μM). After a two-hour incubation, an equal volume of RPMI supplemented with 20% heat-inactivated FBS was added, and cells were incubated for another 48 hours. SEAP activity was accessed using the QUANTI-Blue™ Solution (Invivogen) to evaluate type I interferon protein levels: 150 μl of the colorimetric reagent was added to 50 μl of cell supernatant for 30 min, 37° C., followed by absorption measurement at 655 nm.

### Example 6: Cell Viability

THP1 cells (ATCC, TIB-202) were maintained in RPMI growth medium supplemented with 2-mercaptoethanol, 10% FBS and ciprofloxacin hydrochloride in a humidified atmosphere of 5% CO₂ and 95% air at 37° C. Cells were seeded in 96-well plates at a density of 30,000 cells/well. To generate M2 polarized macrophages, cells were treated first with 185 ng/mL PMA for 6 hours alone and then 20 ng/mL of IL-4 and IL-13 were added for another 16 hours of treatment. At the completion of polarization, the medium was replaced with DMEM without FBS, pH 6.4, containing increasing amounts of pHLIP(Laa)-STINGa. After a three-hour incubation, an equal volume of RPMI supplemented with 20% FBS was added. Cell viability was assessed after 48 hours using the CellTiter 96 AQᵤₑₒᵤₛ One Solution Cell Proliferation Assay (Promega); the colorimetric reagent was added to cells for one hour, followed by absorption measurement at 490 nm.

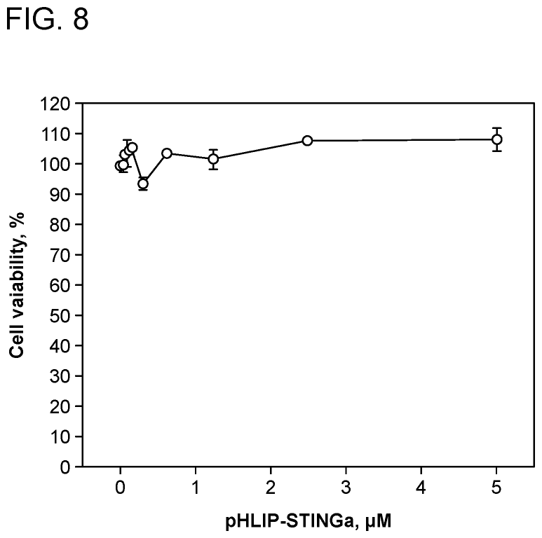

The results are provided in FIG. 8.

### Example 7: Treatment of Mice

For the treatment of CT26 tumors, 5×10⁴ CT26 murine colorectal cancer cells (ATCC, CRL-2638) were injected subcutaneously (SQ) in 100 μl of growth medium into the right flank of female Balb/c mice or athymic female nude mice (strain Hsd Athymic Nude-Foxn1nu) ranging in age from 7 to 9 weeks (both from Envigo RMS, Inc). On day 1, when tumors reached size of 100 mm³ (“small tumors”) or 400-700 mm³ (“large tumors”), mice were randomized into groups, body weight was measured and agents including pHLIP(Laa)-STINGa, pHLIP(Daa)-STINGa, pHLIP(Laa), STINGa (diABZI) or vehicle were given as a single intraperitoneal (IP) or intravenous (IV) injection. Vehicle, pHLIP(Laa), STINGa (diABZI) and pHLIP(Laa)-STINGa were given as a single IP injection of 100 μM 300 μl. pHLIP(Daa)-STINGa was given as a single IV injection of 200 μM 150 μl. The compounds were dissolved in DMSO as a stock solution and transferred to 20% PEG400 in saline containing 0.9% sodium chloride (vehicle). The residual DMSO in the final solution injected into animals was less than 2%. Tumor volume and body weight were measured 3 times per week throughout the study. Measurements of tumors were performed using calipers, and the tumor volume (V) was calculated with the formula:

$$V = 0.52 \cdot L \cdot {W}^{2}$$

where L is the length and W is the width of the measured tumor. Mice were removed from the study and euthanized when the tumor volume was greater than 2000 mm³.

Mice in the pHLIP-STINGa treated group, which stayed tumor-free, were re-challenged with tumor cells injected into the opposite flank on day 61 after a single injection of pHLIP-STINGa. Tumor-free mice were kept for additional 40 days (total of 100 days after the treatment with pHLIP-STINGa) and most of them were euthanized. Five tumor-free mice on day 101 received another SQ injection of 10⁵ 4T1 murine breast cancer cells (ATCC, CRL-2539) into their right flanks. Also, a control group of female Balb/c mice received 10⁵ 4T1 cancer cells into the right flank and tumor growth was compared between groups.

For the treatment of 4T1 triple negative breast tumors, 10⁵ 4T1 murine breast cancer cells (ATTC, CRL-2539) were injected SQ in 100 μl of growth medium into the right flank of Balb/c female mice ranging in age from 7 to 9 weeks (Envigo). On day 1, when tumors reached 100 mm³ in volume, the body weight was measured and mice were randomized into four groups. On day 1 mice from groups #2 and #4 received a single IP injection of pHLIP(Laa)-STINGa (100 μM 300 μl). On days 4, 9, 14 mice from groups #3 and #4 received three IP injections of anti-mouse PD-1 antibody (BioCell, CD279), 250 μg/mouse per injection. Mice from the control group (group #1) did not receive any treatment. Tumor volumes and body weight were measured 3 times per week, and mice were euthanized when the tumor volume was greater than 1500 mm³.

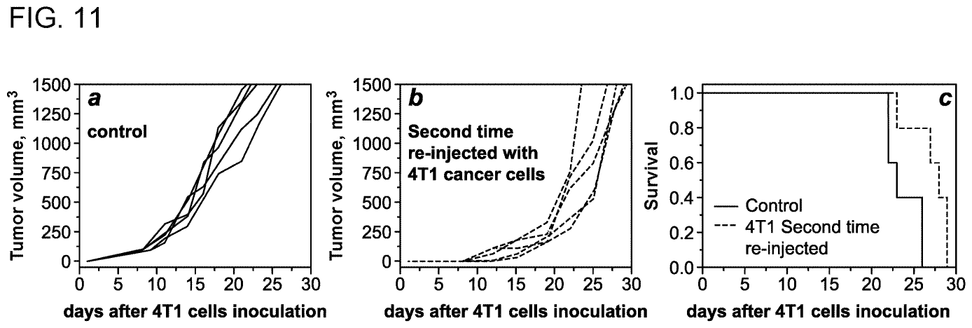

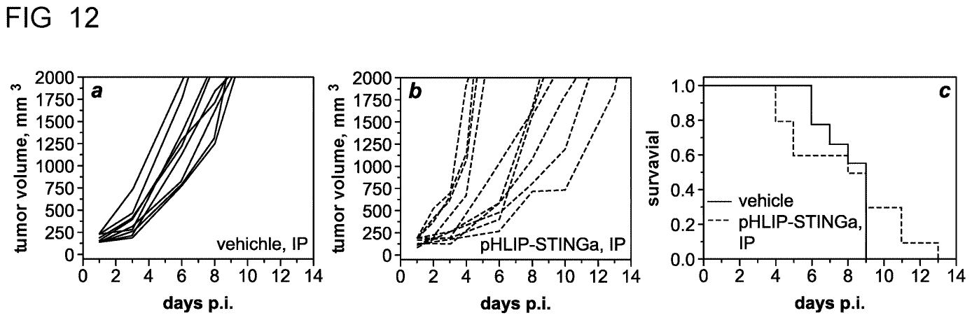

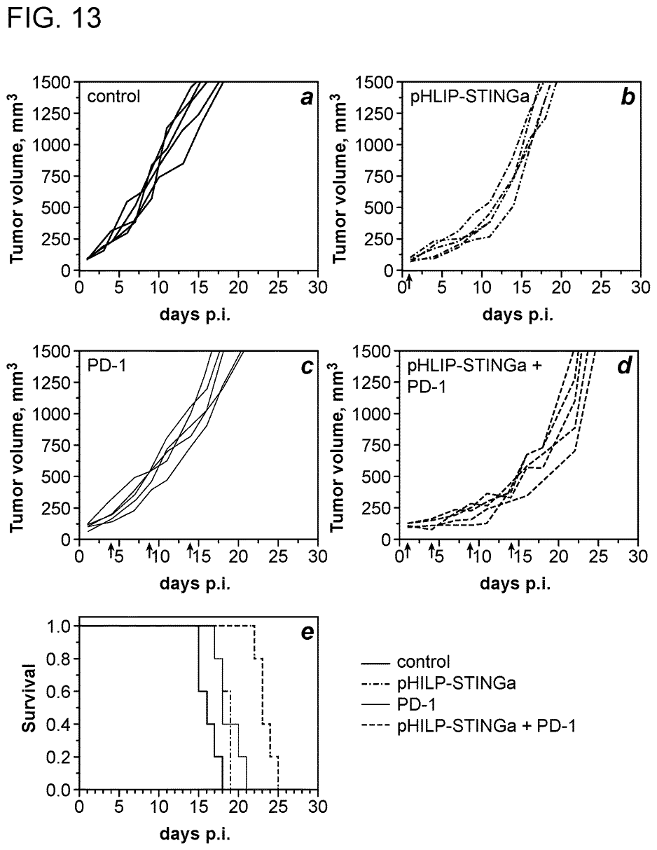

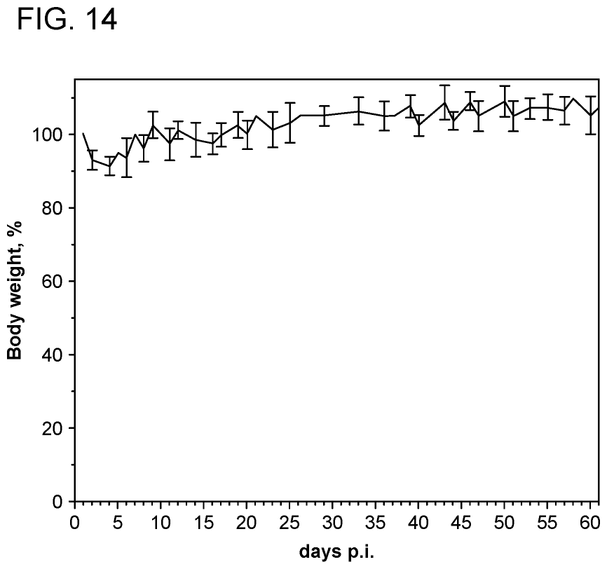

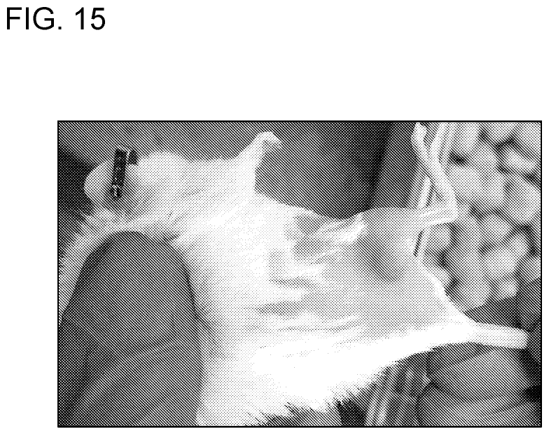

The results are provided in FIGS. 11-15.

### Example 8: ELISA on Blood and Tumor Samples

To establish levels of cytokines in blood and tumor samples, 5×10⁴ CT26 cancer cells were injected SQ in 100 μl of growth medium into the right flank of female Balb/c mice. When tumors reached 150-250 mm³ in volume, the mice received a single IV or IP injection pHLIP(Laa)-STINGa or pHLIP(Daa)-STINGa, STINGa or no injections. Animals were euthanized at 4, 16 and 24 hours post-injection, blood and tumors were collected. Blood samples were kept for 40 min at RT, centrifuged at 5000 g for 20 min at +4° C. and supernatant (serum) was collected. Tumors were frozen in liquid nitrogen. Both, the serum and tumor tissue samples were kept at −80° C. until further processing and analysis. The tumor samples were processed while on ice, using a bullet blender (Next Advance) with 1 mm diameter zirconium silicate beads (Next Advance). The supernatant of the processed tumors was used for enzyme-linked immunoassay (ELISA) assays. Matched antibody pair kits for mouse tissue necrosis factor alpha (TNF-α) (Sino Biological), mouse interleukin 6 (IL-6) (Abcam), and a pre-coated plate for mouse IFN-β (PBL Assay Science) were used. ELISA assays were performed using the serum and tumor samples. For TNF-α the capture antibody was diluted in phosphate buffer saline (PBS) (Sigma-Aldrich), and for IL-6 the capture antibody was diluted in coating buffer (Abcam). The diluted capture antibodies were incubated in the plates overnight at +4° C. and washed the next day with PBS/Tween washing buffer (Sigma-Aldrich). The TNF-α plate was blocked using 2% bovine serum albumin (BSA) (Thermo Scientific) in washing buffer, while the IL-6 and IFN-β plates are blocked using dilution buffers from the corresponding kit. Blocking was done for 2 hours at RT on an orbital shaker at 200 rpm. After blocking, the plates were washed and then incubated with the diluted tumor and serum samples along with the corresponding standard solutions for each ELISA kit. The samples were incubated for 2 hours at RT on an orbital shaker at 200 rpm. The TNF-α and IFN-β plates were incubated for 1 hour at RT with a diluted detection antibody conjugated with horseradish peroxidase (HRP). The IL-6 plate was incubated for 1 hour at RT with a diluted detection antibody conjugated with biotin followed by incubation for 1 hour at RT with the diluted HRP-streptavidin conjugate (Abcam). All plates were washed and incubated with 3,3′,5,5′-tetramethylbenzidine (TMB) (Invitrogen) and peroxide solution mixed at a ratio of 1:1 (Thermo Scientific) for up to 20 min, then stop solution (10% H₂SO₄) was added to the plates. The signal from the wells was quantified by absorbance measured at 450 nm using a Bio-Rad iMark microplate reader. Different dilution schemes were tested in duplicate and antibody standards were used to plot calibration curves.

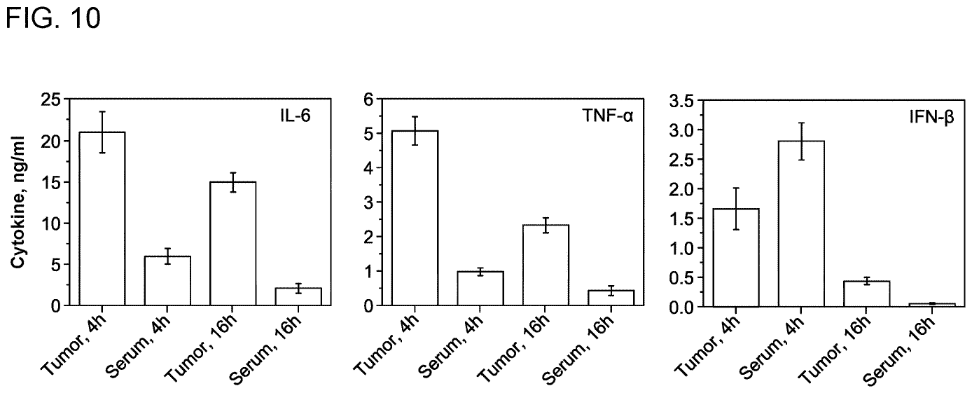

The results are provided in FIG. 10.

### Example 9: Biodistribution, PK and Imaging

For pharmacokinetics (PK), biodistribution and pH imaging studies, 5×10⁴ of CT26 cancer cells were injected SQ in 100 μl of growth medium into the right flank of female Balb/c mice and tumors were grown until they reached 150-200 mm³ in volume. For PK and biodistribution studies, a single tail vein injection of 200 μM 100 μl of ICG-pHILIP (Laa)-STINGa was performed. Animals were euthanized at 2, 4, 24, 48, 72 and 96 hours post-injection, blood was collected in K₂ EDTA vacutainer blood collection tubes (BD), and necropsy was performed immediately after euthanization. Blood, tumors and major organs (kidney, liver, spleen, pancreas, lung, heart, large and small intestines, bone, muscle, brain) were collected, and imaged ex vivo immediately after necropsy. Blood (150 μl) was imaged in 96-well plate with black bottom and walls. The zero-time point (0 min) was obtained by imaging of ICG-pHLIP-STINGa diluted in blood collected from a control mouse that did not receive any injection (the dilution was made based on the assumption that a mouse contains 80 ml/kg of blood). The fluorescence at zero-time point was taken as 100% and fluorescence recorded at 2, 4, 24 and 48 hrs p.i. were calculated as a percentage of zero-time point signal. The points were fitted using single exponential decay function to establish half-life time.

For in vivo imaging, Balb/c and athymic nude mice were given single IP (200 μM 100 μl) or IP (300 μM 150 μl) injections of ICG-pHLIP(Laa)-STINGa when tumors reached 150-250 mm³, and in vivo imaging was performed at 1, 2, 4, 24, 40-48, 74, 100, 170 and 195 hrs p.i.

For pH imaging studies mice were separated into 2 groups. On day 1, mice from group #1 received a single IP injection of pHLIP(Laa)-STINGa (100 μM 300 μl), while mice from the control group #2 did not receive any treatment. On day 3, mice from groups #1 and 2 received a single IP injection of 50 μM 100 μl of the acidity imaging probe ICG-pHLIP (Iris Biotech, GmbH). On day 4 (or 24 hours after ICG-pHLIP injection) all animals were euthanized, tumors were collected, cut in half and imaged.

The in vivo and ex vivo bright field and near-infrared fluorescent imaging was performed using a Stryker 1588 AIM endoscopic system with L10 AIM Light Source (808 nm excitation and collection of light in the range of approximately of 815 to 850 nm), and a 1588 AIM Camera using a 10 mm scope. The lens was kept at a fixed distance from the surface of the organs, within an enclosed (light-protected) area. The imaging was performed at three different settings. The digital images of organs were saved in the green channel, transferred into 8-bit files and processed using ImageJ program. A threshold was set from pixel intensity in the range from 1 to 255, leaving out the background with pixel intensity 0. Brightfield images were used to establish the borders of the organs and tumors. The calculated total fluorescence intensity and total area of each organ were used to calculate the mean organ fluorescence.

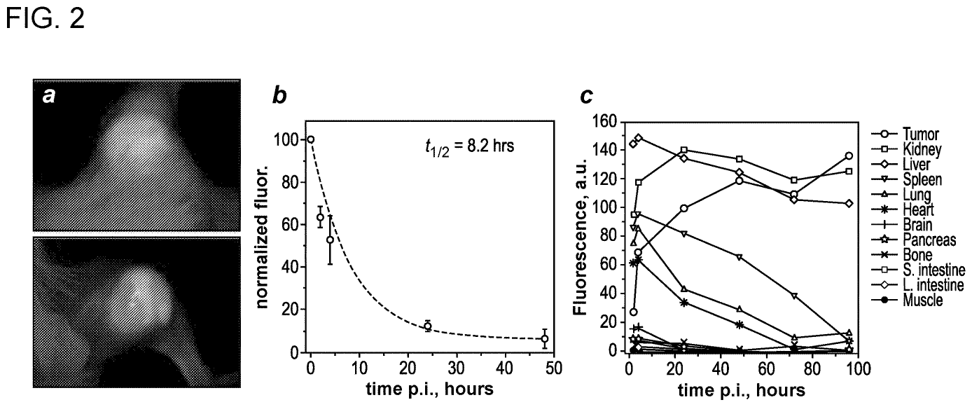

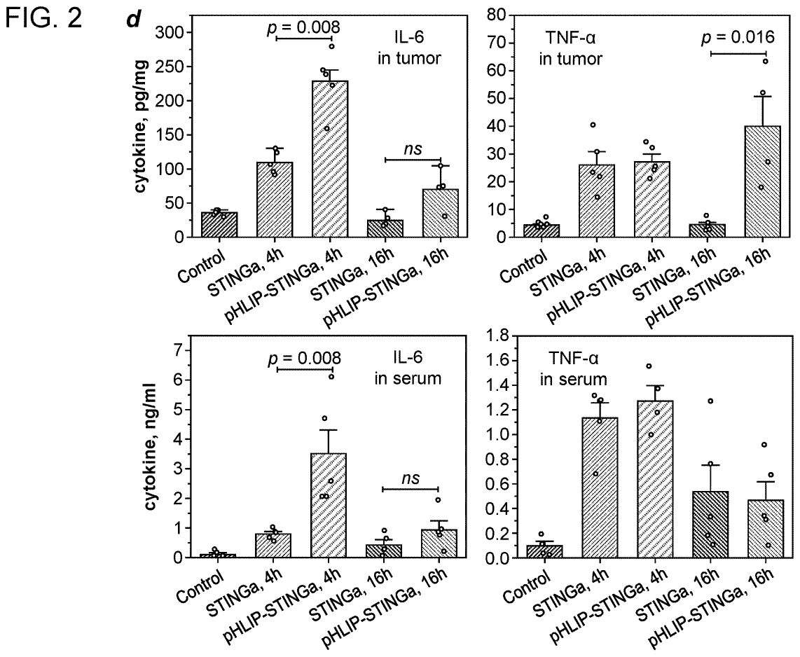

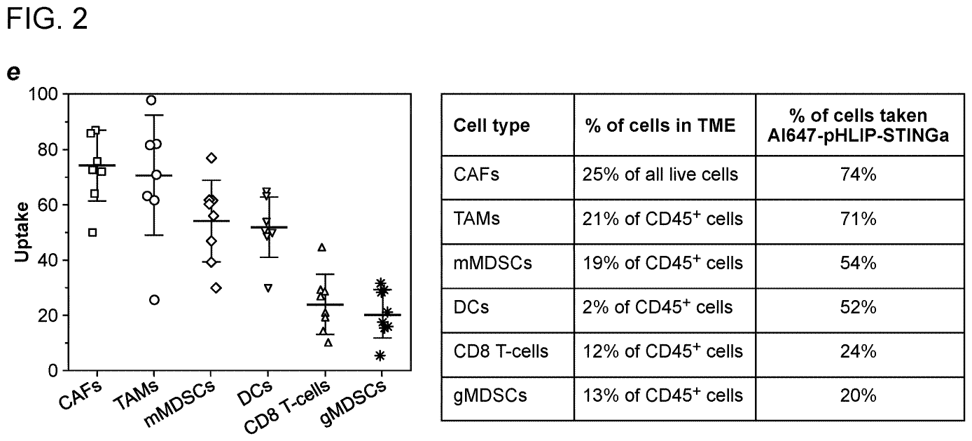

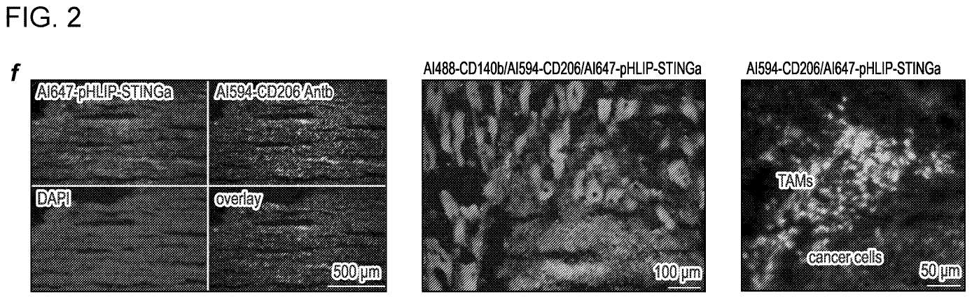

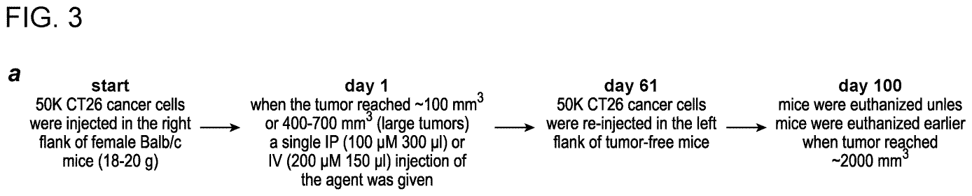

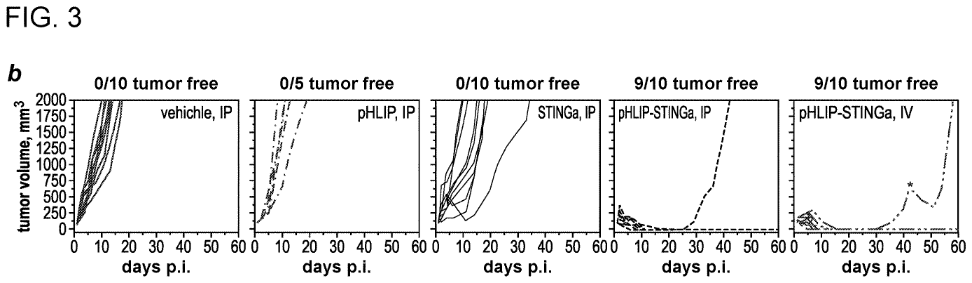

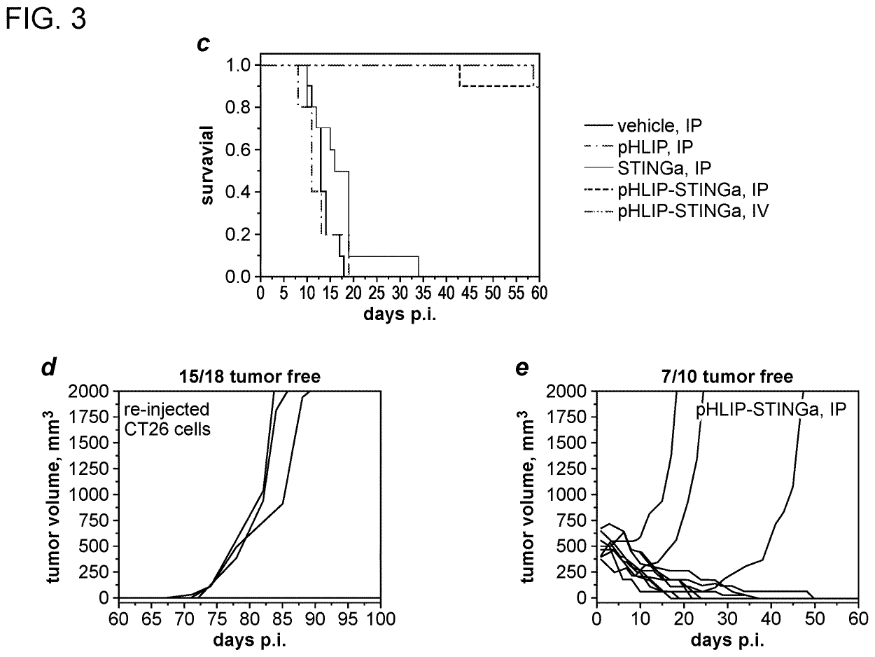

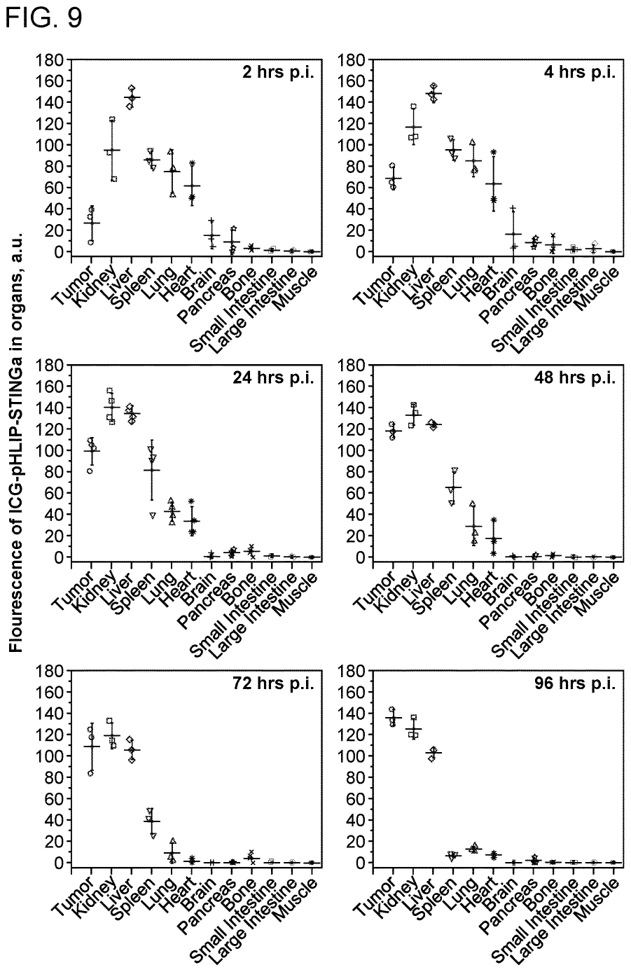

The results from the studies are provided in FIGS. 2, 3, and 9.

### Example 10: FACS Analysis

Uptake of A1647-pHLIP(Laa)-STINGa by tumor cells within CT26 tumors was analyzed by fluorescence activated cell sorting (FACS) analysis. 3×10⁵ CT26 cancer cells were injected SQ in 100 μl of 0% matrigel into the flank of female Balb/c mice (age 8 to 12 weeks). When tumors reached 150-250 mm³ in volume mice were separated into two groups. Eight mice from the treated group #1 received a single IP injection of A1647-pHLIP(Laa)-STINGa (300 μM 100 μl), and five mice from the control group #2 received single IP injection of vehicle (1% DMSO in PBS). 24 hours later all animals were euthanized and tumors were collected for processing. Tumor stroma and immune cells were identified using the following markers:
CD4 T cells: CD45⁺, CD3⁺, CD11b⁻, CD4⁺, CD8⁻
CD8 T cells: CD45⁺, CD3⁺, CD11b⁻, CD4⁻, CD8⁺
Treg: CD45⁺, CD3⁺, CD11b⁻, CD4⁺, CD25⁺, FoxP3⁺
mMDSC: CD45⁺, CD3⁻, CD11b⁺, F4/80⁻, Ly6C^{high}, Ly6G⁻
gMDSC: CD45⁺, CD3⁻, CD11b⁺, F4/80⁻, Ly6C^{low}, Ly6G⁺
M1: CD45⁺, CD11b⁺, F4/80⁺, CD206⁻
M2: CD45⁺, CD11b⁺, F4/80⁺, CD206⁺
DC: CD45⁺, CD11b⁺, CD11c⁺, MHCII⁻, F4/80⁻
CAF: CD3⁻, CD45⁻, CD140b⁺

The staining was performed for tumor samples, Fluorescence Minus One (FMO) controls and Single Color Controls (SCC) as indicated below:
Tumor: Live/Dead, CD45, CD3, CD4, CD8, CD25, FoxP3, CD11b, F4/80, Ly6C, Ly6G, CD206, CD11c, MHCII, CD140b
Tumor-FMO: CD3, CD25, FoxP3, F4/80, Ly6C, Ly6G, CD206, CD11c, MHCII, CD140b
SCC: Unstained, CD45, CD3, CD4, CD8, CD25, FoxP3, CD11b, F4/80, Ly6C, Ly6G, CD206, CD11c, MHCII, CD140b, Live/Dead

The following anti-mouse antibodies were used: CD45-APC-Fire750 clone 30-F11, CD8a-BV650 clone 53-6.7, CD25-BV605 clone PC61, F4/80-PE-Dazzle-594 clone BM8, Ly-6C-FITC clone HK1.4, Ly-6G-BV785 clone 1A8, CD206-BV421 clone C068C2, CD140b-PE clone APB5, CD11c-BV711 clone N418, I-A/I-E-PE/Cy7 clone M5/114.15.2 from BioLegend; CD3e-BUV496 clone 145-2C11, CD4-BUV395 clone GK1.5; CD11b-BUV737 clone M1/70 from BD Biosciences, FoxP3 PerCP-Cy5.5 clone FJK-16s from Thermo Fisher and Live/Dead Aqua-V500 from Life Technologies. The procedure for tumor processing was the following: tumor samples were dissociated according to the manufacturer's instructions using the gentleMACS™ protocol “Tumor Dissociation Kit”. Samples were filtered through a 70 μm cell strainer and rinsed twice in PBS/2.5% FBS buffer and total sample volumes were measured. Single cell suspensions were prepared in PBS pH 7.4 at 1×10⁷ cells/mL and placed into individual wells of a 96-well plate and kept on ice. All incubation steps were carried out protected from light. The washing was performed by spinning the plate at 300×g (or 400×g) for 3 minutes and discarding the supernatant. Live/Dead reagent was added to each sample and incubated at 4° C. for 15 minutes followed by washing. Fc-block (Mu TruStain FcX/anti-FcγRIV, Biolegend) diluted in Staining Buffer (BD) was added to the samples and incubated for 10 min at 4° C. followed by addition of cell surface antibodies diluted in Staining Buffer supplemented with Brilliant Stain Buffer Plus (BD) for 30 min at 4° C. and consequent washing. Cells were fixed in FoxP3 Fix/Perm solution and incubated for 30 min at room temperature followed washing. For single color controls, one drop of Ultra Comp Beads (Thermo Fisher) was added to each single-color control well. For Live/Dead controls one drop of ArC Amine Reactive Compensation Bead (Life Technologies) were added followed by addition of each antibody to appropriate well. Isotype control-A1647 clone MOPC-21 (BioLegend) was used for Alexa47 channel, where A1647-pHLIP-STINGa was imaged. The incubation steps were followed by washing steps. The number of cells in the control and treated groups and the cellular uptake of A1647-pHLIP-STINGa was established.

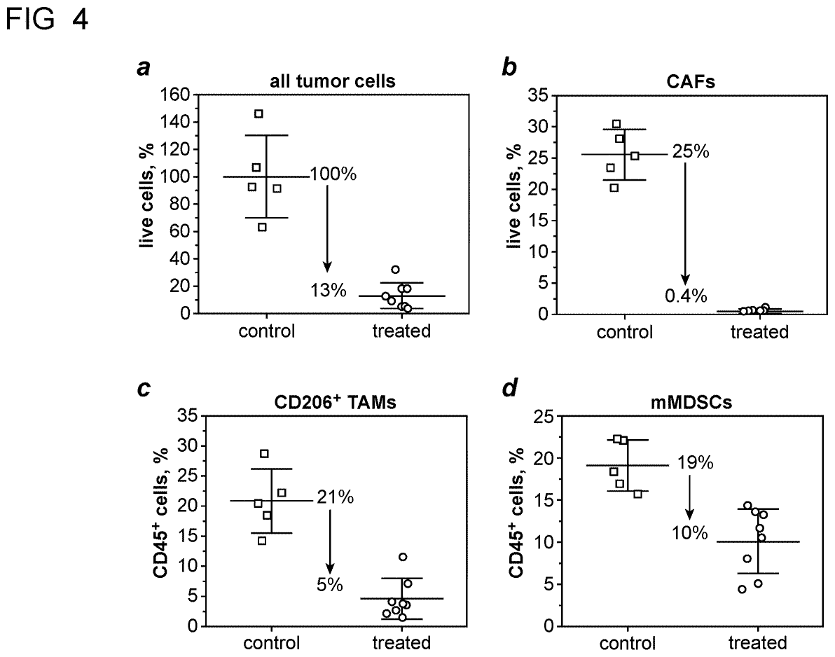

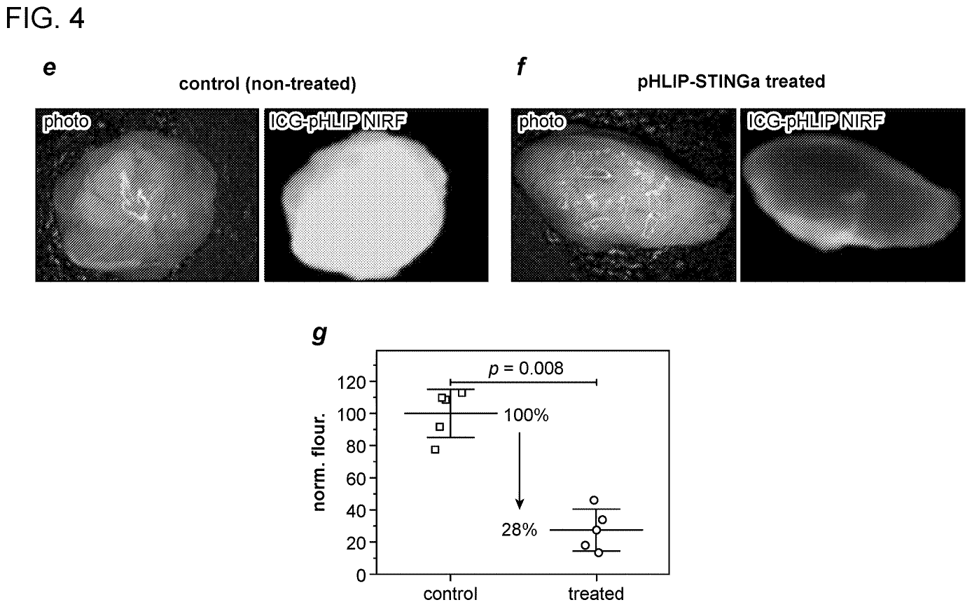

The results are provided in FIG. 4.

### Example 11: Immuno-Histochemistry and Imaging

For immunohistochemistry analysis 5×10⁴ CT26 cancer cells were injected SQ in 100 μl of growth medium into the right flank of female BALB/c mice. When the tumors reached 150-250 mm³ in volume, mice received a single IP injection of A1647-pHLIP(Laa)-STINGa (300 μM 100 μl). Tumors were cryo-sectioned using a ThermoFisher HM525 NX to make 10-20 μm sections. Sections were stained with fluorescent antibodies, CD206-AL594 (BioLegend), CD68-AL594 (BioLegend), CD140b-AL488 (Invitrogen) and 4′,6-diamidino-2-phenylindole (DAPI) (Sigma-Aldrich) or hematoxylin and eosin (H&E) using hematoxylin 7211 (ThermoFisher) and eosin Y (Poly Scientific). Antibody-stained sections were dried in air for 10 min, then washed with distilled water for 2 min followed by fixation in 4% paraformaldehyde 37% (Sigma-Aldrich) for 12 min, washing with Dulbecco's phosphate buffered saline (DPBS) (Sigma-Aldrich) for 5 min and drying in air for 10 min. A cover slide was placed on a layer of petroleum jelly (Equate), which was applied to the slide around the tissue. Sections were incubated with blocking buffer containing 5% of 10% BSA (ThermoFisher) for 2 hours at RT followed by washing. Sections were treated with antibody in blocking buffer for 2 hours at RT, followed by washing. A coverslip is mounted on top of the tissue using organo/limonene mount. Imaging of the tissue sections were performed on an EVOS F1 Auto 2 fluorescence inverted microscope using 10×, 20× and 40× objectives in brightfield and fluorescent modes with appropriate filters.

### Example 12: Statistical Analysis

The Kolmogorov-Smirnov two-tailed nonparametric test was used to establish p-levels. Log rank (by weighting all time points the same), Breslow method (by weighting all time points by the number of cases at risk at each time point) and Tarone-Ware method (by weighting all time points by the square root of the number of cases at risk at each time point) were used to establish p-levels for survival plots.

The patent and scientific literature referred to herein establishes the knowledge that is available to those with skill in the art. All United States patents and published or unpublished United States patent applications cited herein are hereby incorporated by reference. All published foreign patents and patent applications cited herein are hereby incorporated by reference. Genbank and NCBI submissions, indicated by accession number, cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A composition comprising the following structure: 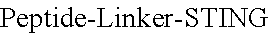
wherein Peptide is a pHLIP® peptide,
wherein Linker is a cleavable linker;
wherein “STING” is a STING agonist selected from the group consisting of diABZI, ADU-S100 (MIW815), MK-1454, MK-2118, SB11285, GSK3745417, BMS-986301, BI1387446, E7766, TAK-676, SNZ281, IMSA-101, IMSA-201, SYNB1891, JNJ-4412, JNJ-6196, BI-STING, GSK532, TTI-10001, CRD5500, LB-061, ALG-031048, RVU312-4787, TAK-500, CL656, CDK-002, CF501, CF502, CF503, CF504, CF505, CF506, CF507, CF508, CF509, CF510, CF511, and derivatives thereof; and
wherein each “-” is a covalent bond.

2. (canceled)

3. The composition of claim 1, wherein the pHLIP® peptide comprises the sequence ADDQNPWRAYLDLLFPTDTLLLDLLWXG (SEQ ID NO: 220), wherein “X” is a functional group for conjugation purposes, selected from lysine (Lys), cysteine (Cys), Azido-containing amino acid or other modified amino acids.

4. (canceled)

5. The composition of claim 1, wherein the pHLIP® peptide comprises a sequence selected from the group consisting of any one of SEQ ID NOs. 1-219.

6.-25. (canceled)

26. A composition comprising the following structure: 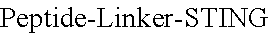
wherein Peptide is a pHLIP® peptide;
wherein Linker is a cleavable linker;
wherein STING is a STING agonist; and
wherein each “-” is a covalent bond.

27. The composition of claim 26, wherein the pHLIP® peptide comprises the sequence ADQDNPWRAYLDLLFPTDTLLLDLLWCG (SEQ ID NO: 218).

28. The composition of claim 26, wherein the pHLIP® peptide comprises the sequence ADDQNPWRAYLDLLFPTDTLLLDLLWCG (SEQ ID NO: 219).

29.-32. (canceled)

33. The composition of claim 26, wherein the STING agonist comprises a cyclic dinucleotide (CDN).

34. The composition of claim 33, wherein the STING agonist comprises a cyclic purine dinucleotide.

35. The composition of claim 26, wherein the STING agonist is a non-nucleotide small molecule.

36. The composition of claim 26, wherein the STING agonist is selected from the group consisting of diABZI, ADU-S100 (MIW815), MK-1454, MK-2118, SB11285, GSK3745417, BMS-986301, BI1387446, E7766, TAK-676, SNZ281, IMSA-101, IMSA-201, SYNB1891, JNJ-4412, JNJ-6196, BI-STING, GSK532, TTI-10001, CRD5500, LB-061, ALG-031048, RVU312-4787, TAK-500, CL656, CDK-002, CF501, CF502, CF503, CF504, CF505, CF506, CF507, CF508, CF509, CF510, CF511, and derivatives thereof.

37. The composition of claim 36, wherein the STING agonist is diABZI, MK-1454, MK-2118, TAK-676, BMS-986301, or BI1387446.

38.-42. (canceled)

43. The composition of claim 26, wherein the Linker comprises a disulfide bond or an acid-labile bond.

44. The composition of claim 43, wherein the Linker is self-immolating.

45. The composition of claim 26, wherein the composition has the following structure:

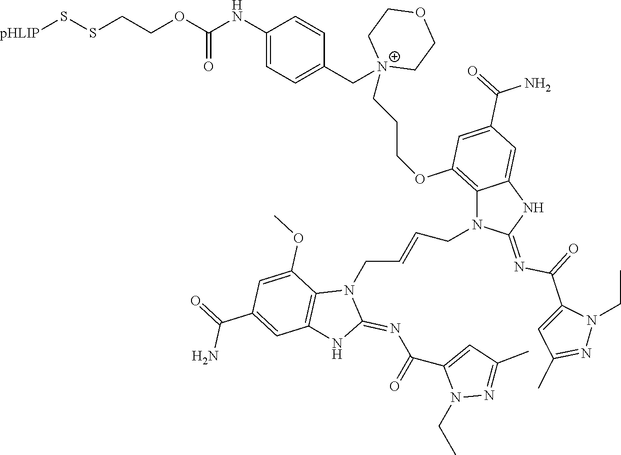

46. A pharmaceutical composition comprising the composition of claim 26 and one or more pharmaceutically acceptable excipients.

47. A method of treating cancer in a subject in need thereof comprising administering the composition of claim 26 to the subject in need thereof.

48. The method of claim 47, wherein the cancer is a solid tumor.

49. The method of claim 47, wherein the composition of claim 26 is injected directly into a tumor mass.

50. The method of claim 47, wherein the composition of claim 26 is systemically administered.

51. The method of claim 47, wherein the subject is a human.


SEQUENCE LISTING (221 sequences)

SEQ ID NO: 1-2 - skipped (2 entries)

SEQ ID NO: 3 - protein, 35 aa, synthetic construct
AEQNPIYWARYADWLFTTPLLLLDLALLVDADEGT

SEQ ID NO: 4 - protein, 7 aa, synthetic construct
AEQNPIY

SEQ ID NO: 5 - protein, 22 aa, synthetic construct
WARYADWLFTTPLLLLDLALLV

SEQ ID NO: 6 - protein, 6 aa, synthetic construct
DADEGT

SEQ ID NO: 7 - protein, 36 aa, synthetic construct
GGEQNPIYWARYADWLFTTPLLLLDLALLVDADEGT

SEQ ID NO: 8 - protein, 35 aa, synthetic construct; identical to SEQ ID NO: 3

SEQ ID NO: 9 - protein, 38 aa, synthetic construct
AAEQNPIYWARYADWLFTTPLLLLDLALLVDADEGTCG

SEQ ID NO: 10 - skipped

SEQ ID NO: 11 - protein, 38 aa, synthetic construct
GGEQNPIYWARYADWLFTTPLLLLDLALLVDADEGTCG

SEQ ID NO: 12 - protein, 37 aa, synthetic construct
  source 1..37: note = Acetylated N-terminus
ACEQNPIYWARYADWLFTTPLLLLDLALLVDADEGTG

SEQ ID NO: 13 - protein, 36 aa, synthetic construct
ACEQNPIYWARYADWLFTTPLLLLDLALLVDADEGT

SEQ ID NO: 14 - protein, 36 aa, synthetic construct
AKEQNPIYWARYADWLFTTPLLLLDLALLVDADEGT

SEQ ID NO: 15 - protein, 37 aa, synthetic construct
  source 1..37: note = Acetylated N-terminus
AKEQNPIYWARYADWLFTTPLLLLDLALLVDADEGTG

SEQ ID NO: 16 - protein, 39 aa, synthetic construct
AAEQNPIYWARYADWLFTTPLLLLDLALLVDADEGTKCG

SEQ ID NO: 17 - protein, 36 aa, synthetic construct
AKEQNPIYWARYADWLFTTPLLLLDLALLVDADECT

SEQ ID NO: 18 - protein, 37 aa, synthetic construct
ACEQNPIYWARYANWLFTTPLLLLNLALLVDADEGTG

SEQ ID NO: 19 - protein, 36 aa, synthetic construct
ACEQNPIYWARYANWLFTTPLLLLNLALLVDADEGT

SEQ ID NO: 20 - protein, 37 aa, synthetic construct
ACEQNPIYWARYAKWLFTTPLLLLKLALLVDADEGTG

SEQ ID NO: 21 - protein, 36 aa, synthetic construct
GGEQNPIYWARYADWLFTTPLLLLDLALLVNANQGT

SEQ ID NO: 22 - protein, 36 aa, synthetic construct
AAEQNPIYWARYADWLFTTPLLLLALALLVDADEGT

SEQ ID NO: 23 - protein, 39 aa, synthetic construct
  source 1..39: note = Acetylated N-terminus
AAEQNPIYWARYADWLFTTPLLLLELALLVDADEGTKCG

SEQ ID NO: 24 - protein, 36 aa, synthetic construct
AAEQNPIYWARYAAWLFTTPLLLLDLALLVDADEGT

SEQ ID NO: 25 - protein, 36 aa, synthetic construct
AAEQNPIYWARYADWLFTTALLLLDLALLVDADEGT

SEQ ID NO: 26 - protein, 36 aa, synthetic construct
AAEQNPIYWARYADWLFTTPLLLLELALLVDADEGT

SEQ ID NO: 27 - protein, 36 aa, synthetic construct
AAEQNPIYWARYAEWLFTTPLLLLDLALLVDADEGT

SEQ ID NO: 28 - protein, 39 aa, synthetic construct
AAEQNPIIYWARYADWLFTDLPLLLLDLLALLVDADEGT

SEQ ID NO: 29 - protein, 37 aa, synthetic construct
GEQNPIYWAQYADWLFTTPLLLLDLALLVDADEGTCG

SEQ ID NO: 30 - protein, 38 aa, synthetic construct
GGEQNPIYWARYADWLFTTPLLLDLLALLVDADEGTCG

SEQ ID NO: 31 - protein, 38 aa, synthetic construct
GGEQNPIYWARYADWLFTTPLLLLLDALLVDADEGTCG

SEQ ID NO: 32 - protein, 38 aa, synthetic construct
GGEQNPIYWARYDAWLFTTPLLLLDLALLVDADEGTCG

SEQ ID NO: 33 - protein, 38 aa, synthetic construct
GGEQNPIYWARYAWDLFTTPLLLLDLALLVDADEGTCG

SEQ ID NO: 34 - protein, 36 aa, synthetic construct
AAEQNPIYWARYADWLFTTGLLLLDLALLVDADEGT

SEQ ID NO: 35 - protein, 37 aa, synthetic construct
DDDEDNPIYWARYADWLFTTPLLLLHGALLVDADECT

SEQ ID NO: 36 - protein, 36 aa, synthetic construct
DDDEDNPIYWARYADWLFTTPLLLLHGALLVDADET

SEQ ID NO: 37 - protein, 38 aa, synthetic construct
DDDEDNPIYWARYAHWLFTTPLLLLHGALLVDADEGCT

SEQ ID NO: 38 - protein, 37 aa, synthetic construct
CDDDEDNPIYWARYAHWLFTTPLLLLHGALLVDADET

SEQ ID NO: 39 - protein, 37 aa, synthetic construct
DDDEDNPIYWARYAHWLFTTPLLLLHGALLVDADEGT

SEQ ID NO: 40 - protein, 37 aa, synthetic construct
DDDEDNPIYWARYAHWLFTTPLLLLHGALLVNADECT

SEQ ID NO: 41 - protein, 37 aa, synthetic construct
DDDEDNPIYWARYAHWLFTTPLLLLHGALLVNADEGT

SEQ ID NO: 42 - protein, 37 aa, synthetic construct
DDDEDNPIYWARYAHWLFTTPLLLLHGALLVNANECT

SEQ ID NO: 43 - protein, 37 aa, synthetic construct
DDDEDNPIYWARYAHWLFTTPLLLLHGALLVNANEGT

SEQ ID NO: 44 - protein, 34 aa, synthetic construct
AEQNPIYWARYADFLFTTPLLLLDLALLVDADET

SEQ ID NO: 45 - protein, 35 aa, synthetic construct
AEQNPIYFARYADWLFTTPLLLLDLALLVDADEGT

SEQ ID NO: 46 - protein, 34 aa, synthetic construct
AEQNPIYFARYADFLFTTPLLLLDLALLWDADET

SEQ ID NO: 47 - protein, 32 aa, synthetic construct
AKEDQNPYWARYADWLFTTPLLLLDLALLVDG

SEQ ID NO: 48 - protein, 32 aa, synthetic construct
ACEDQNPYWARYADWLFTTPLLLLDLALLVDG

SEQ ID NO: 49 - protein, 32 aa, synthetic construct
AEDQNPYWARYADWLFTTPLLLLDLALLVDCG

SEQ ID NO: 50 - protein, 32 aa, synthetic construct
AEDQNPYWARYADWLFTTPLLLLELALLVECG

SEQ ID NO: 51 - protein, 32 aa, synthetic construct
AKEDQNDPYWARYADWLFTTPLLLLDLALLVG

SEQ ID NO: 52 - protein, 30 aa, synthetic construct
AKEDQNPYWRAYADLFTPLTLLDLLALWDG

SEQ ID NO: 53 - protein, 31 aa, synthetic construct
AEDQNPYWARYADWLFTTPLLLLELALLVCG

SEQ ID NO: 54 - protein, 30 aa, synthetic construct
ACEDQNPYWRAYADLFTPLTLLDLLALWDG

SEQ ID NO: 55 - protein, 27 aa, synthetic construct
ACDDQNPWRAYLDLLFPTDTLLLDLLW

SEQ ID NO: 56 - protein, 28 aa, synthetic construct
AKDDQNPWRAYLDLLFPTDTLLLDLLWC

SEQ ID NO: 57 - protein, 27 aa, synthetic construct
ACEEQNPWRAYLELLFPTETLLLELLW

SEQ ID NO: 58 - protein, 25 aa, synthetic construct
ACDDQNPWARYLDWLFPTDTLLLDL

SEQ ID NO: 59 - protein, 24 aa, synthetic construct
CDNNNPWRAYLDLLFPTDTLLLDW

SEQ ID NO: 60 - protein, 24 aa, synthetic construct
CEEQQPWAQYLELLFPTETLLLEW

SEQ ID NO: 61 - protein, 24 aa, synthetic construct
CEEQQPWRAYLELLFPTETLLLEW

SEQ ID NO: 62 - protein, 37 aa, synthetic construct
CDDDDDNPNYWARYANWLFTTPLLLLNGALLVEAEET

SEQ ID NO: 63 - protein, 36 aa, synthetic construct
CDDDDDNPNYWARYAPWLFTTPLLLLPGALLVEAEE

SEQ ID NO: 64 - protein, 34 aa, synthetic construct
  source 1..34: note = Acetylated N-terminus, Amidated C terminus
TEDADVLLALDLLLLPTTFLWDAYRAWYPNQECA

SEQ ID NO: 65 - protein, 20 aa, synthetic construct
AEQNPIYWARYADWLFTTPL

SEQ ID NO: 66 - protein, 21 aa, synthetic construct
AEQNPIYWARYADWLFTTPCL

SEQ ID NO: 67 - protein, 21 aa, synthetic construct
ACEQNPIYWARYADWLFTTPL

SEQ ID NO: 68 - protein, 20 aa, synthetic construct
AEQNPIYFARYADWLFTTPL

SEQ ID NO: 69 - protein, 22 aa, synthetic construct
AEQNPIYFARYADLLFPTTLAW

SEQ ID NO: 70 - protein, 22 aa, synthetic construct
AEQNPIYWARYADLLFPTTLAF

SEQ ID NO: 71 - protein, 22 aa, synthetic construct
AEQNPIYWARYADLLFPTTLAW

SEQ ID NO: 72 - protein, 21 aa, synthetic construct
KEDQNPWARYADLLFPTTLAW

SEQ ID NO: 73 - protein, 20 aa, synthetic construct
KEDQNPWARYADLLFPTTLW

SEQ ID NO: 74 - protein, 22 aa, synthetic construct
ACEDQNPWARYADLLFPTTLAW

SEQ ID NO: 75 - protein, 24 aa, synthetic construct
ACEDQNPWARYADWLFPTTLLLLD

SEQ ID NO: 76 - protein, 22 aa, synthetic construct
ACEEQNPWARYAELLFPTTLAW

SEQ ID NO: 77 - protein, 24 aa, synthetic construct
ACEEQNPWARYAEWLFPTTLLLLE

SEQ ID NO: 78 - protein, 25 aa, synthetic construct
ACEEQNPWARYLEWLFPTETLLLEL

SEQ ID NO: 79 - protein, 24 aa, synthetic construct
ACEEQNPQAEYAEWLFPTTLLLLE

SEQ ID NO: 80 - protein, 21 aa, synthetic construct
WARYADWLFTTPLLLLDLALL

SEQ ID NO: 81 - protein, 21 aa, synthetic construct
YARYADWLFTTPLLLLDLALL

SEQ ID NO: 82 - protein, 21 aa, synthetic construct
WARYSDWLFTTPLLLYDLGLL

SEQ ID NO: 83 - protein, 21 aa, synthetic construct
WARYTDWFTTPLLLYDLALLA

SEQ ID NO: 84 - protein, 21 aa, synthetic construct
WARYTDWLFTTPLLLYDLGLL

SEQ ID NO: 85 - protein, 21 aa, synthetic construct
WARYADWLFTTPLLLLDLSLL

SEQ ID NO: 86 - protein, 21 aa, synthetic construct
LLALDLLLLPTTFLWDAYRAW

SEQ ID NO: 87 - protein, 21 aa, synthetic construct
LLALDLLLLPTTFLWDAYRAY

SEQ ID NO: 88 - protein, 21 aa, synthetic construct
LLGLDYLLLPTTFLWDSYRAW

SEQ ID NO: 89 - protein, 21 aa, synthetic construct
ALLALDYLLLPTTFWDTYRAW

SEQ ID NO: 90 - protein, 21 aa, synthetic construct
LLGLDYLLLPTTFLWDTYRAW

SEQ ID NO: 91 - protein, 21 aa, synthetic construct
LLSLDLLLLPTTFLWDAYRAW

SEQ ID NO: 92 - protein, 26 aa, synthetic construct
GLAGLLGLEGLLGLPLGLLEGLWLGL

SEQ ID NO: 93 - protein, 26 aa, synthetic construct
LGLWLGELLGLPLGLLGELGLLGALG

SEQ ID NO: 94 - protein, 20 aa, synthetic construct
WRAYLDLLFPTDTLLLDLLW

SEQ ID NO: 95 - protein, 20 aa, synthetic construct
WLLDLLLTDTPFLLDLYARW

SEQ ID NO: 96 - protein, 18 aa, synthetic construct
WARYLEWLFPTETLLLEL

SEQ ID NO: 97 - protein, 18 aa, synthetic construct
WAQYLELLFPTETLLLEW

SEQ ID NO: 98 - protein, 18 aa, synthetic construct
LELLLTETPFLWELYRAW

SEQ ID NO: 99 - protein, 18 aa, synthetic construct
WELLLTETPFLLELYQAW

SEQ ID NO: 100 - protein, 17 aa, synthetic construct
WLFTTPLLLLNGALLVE

SEQ ID NO: 101 - protein, 17 aa, synthetic construct
WLFTTPLLLLPGALLVE

SEQ ID NO: 102 - protein, 15 aa, synthetic construct
WARYADLLFPTTLAW

SEQ ID NO: 103 - protein, 17 aa, synthetic construct
EVLLAGNLLLLPTTFLW

SEQ ID NO: 104 - protein, 17 aa, synthetic construct
EVLLAGPLLLLPTTFLW

SEQ ID NO: 105 - protein, 15 aa, synthetic construct
WALTTPFLLDAYRAW

SEQ ID NO: 106 - protein, 24 aa, synthetic construct
NLEGFFATLGGEIALWSLVVLAIE

SEQ ID NO: 107 - protein, 22 aa, synthetic construct
EGFFATLGGEIALWSDVVLAIE

SEQ ID NO: 108 - protein, 22 aa, synthetic construct
EGFFATLGGEIPLWSDVVLAIE

SEQ ID NO: 109 - protein, 24 aa, synthetic construct
EIALVVLSWLAIEGGLTAFFGELN

SEQ ID NO: 110 - protein, 22 aa, synthetic construct
EIALVVDSWLAIEGGLTAFFGE

SEQ ID NO: 111 - protein, 22 aa, synthetic construct
EIALVVDSWLPIEGGLTAFFGE

SEQ ID NO: 112 - protein, 21 aa, synthetic construct
ILDLVFGLLFAVTSVDFLVQW

SEQ ID NO: 113 - protein, 21 aa, synthetic construct
WQVLFDVSTVAFLLGFVLDLI

SEQ ID NO: 114 - protein, 34 aa, synthetic construct
AEQNPIYWARYADWLFTTPLLLLDLALLVDADET

SEQ ID NO: 115 - protein, 34 aa, synthetic construct
AEQNPIYWARYAEWLFTTPLLLLELALLVEAEET

SEQ ID NO: 116 - protein, 33 aa, synthetic construct
ADDQNPWRAYLDLLFPDTTDLLLLDLLWDADET

SEQ ID NO: 117 - protein, 33 aa, synthetic construct
AEEQNPWRAYLELLFPETTELLLLELLWEAEET

SEQ ID NO: 118 - protein, 34 aa, synthetic construct
  MOD_RES 13: note = Gamma-carboxyglutamic acid
AEQNPIYWARYAXWLFTTPLLLLDLALLVDADET

SEQ ID NO: 119 - protein, 34 aa, synthetic construct
  MOD_RES 24: note = Gamma-carboxyglutamic acid
AEQNPIYWARYADWLFTTPLLLLXLALLVDADET

SEQ ID NO: 120 - protein, 34 aa, synthetic construct
  MOD_RES 13: note = Gamma-carboxyglutamic acid
  MOD_RES 24: note = Gamma-carboxyglutamic acid
AEQNPIYWARYAXWLFTTPLLLLXLALLVDADET

SEQ ID NO: 121 - protein, 34 aa, synthetic construct; identical to SEQ ID NO: 118
  MOD_RES 13: note = Alpha-aminoadipic acid

SEQ ID NO: 122 - protein, 34 aa, synthetic construct; identical to SEQ ID NO: 119
  MOD_RES 24: note = Alpha-aminoadipic acid

SEQ ID NO: 123 - protein, 34 aa, synthetic construct; identical to SEQ ID NO: 120
  MOD_RES 13: note = Alpha-aminoadipic acid
  MOD_RES 24: note = Alpha-aminoadipic acid

SEQ ID NO: 124 - protein, 34 aa, synthetic construct; identical to SEQ ID NO: 120
  MOD_RES 13: note = Gamma-carboxyglutamic acid
  MOD_RES 24: note = Alpha-aminoadipic acid

SEQ ID NO: 125 - protein, 34 aa, synthetic construct; identical to SEQ ID NO: 120
  MOD_RES 13: note = Alpha-aminoadipic acid
  MOD_RES 24: note = Gamma-carboxyglutamic acid

SEQ ID NO: 126 - protein, 36 aa, synthetic construct; identical to SEQ ID NO: 7

SEQ ID NO: 127 - protein, 36 aa, synthetic construct; identical to SEQ ID NO: 7

SEQ ID NO: 128 - protein, 36 aa, synthetic construct
AAEQNPIYWARYADWLFTTPLLLLDLALLVDADEGT

SEQ ID NO: 129 - protein, 35 aa, synthetic construct; identical to SEQ ID NO: 3

SEQ ID NO: 130 - protein, 35 aa, synthetic construct
AEQNPIYWARYANWLFTTPLLLLNLALLVDADEGT

SEQ ID NO: 131 - protein, 35 aa, synthetic construct
AEQNPIYWARYAKWLFTTPLLLLKLALLVDADEGT

SEQ ID NO: 132 - protein, 36 aa, synthetic construct; identical to SEQ ID NO: 21

SEQ ID NO: 133 - protein, 36 aa, synthetic construct; identical to SEQ ID NO: 22

SEQ ID NO: 134 - protein, 36 aa, synthetic construct; identical to SEQ ID NO: 24

SEQ ID NO: 135 - protein, 36 aa, synthetic construct; identical to SEQ ID NO: 25

SEQ ID NO: 136 - protein, 36 aa, synthetic construct; identical to SEQ ID NO: 26

SEQ ID NO: 137 - protein, 36 aa, synthetic construct; identical to SEQ ID NO: 27

SEQ ID NO: 138 - protein, 39 aa, synthetic construct; identical to SEQ ID NO: 28

SEQ ID NO: 139 - protein, 34 aa, synthetic construct
GEQNPIYWAQYADWLFTTPLLLLDLALLVDADEG

SEQ ID NO: 140 - protein, 35 aa, synthetic construct
GGEQNPIYWARYADWLFTTPLLLDLLALLVDADEG

SEQ ID NO: 141 - protein, 35 aa, synthetic construct
GGEQNPIYWARYADWLFTTPLLLLLDALLVDADEG

SEQ ID NO: 142 - protein, 36 aa, synthetic construct
GGEQNPIYWARYDAWLFTTPLLLLDLALLVDADEGT

SEQ ID NO: 143 - protein, 35 aa, synthetic construct
GGEQNPIYWARYAWDLFTTPLLLLDLALLVDADEG

SEQ ID NO: 144 - protein, 36 aa, synthetic construct; identical to SEQ ID NO: 34

SEQ ID NO: 145 - protein, 34 aa, synthetic construct
DDDEDNPIYWARYADWLFTTPLLLLHGALLVDAD

SEQ ID NO: 146 - protein, 35 aa, synthetic construct
DDDEDNPIYWARYAHWLFTTPLLLLHGALLVDADE

SEQ ID NO: 147 - protein, 33 aa, synthetic construct
CEQNPIYWARYADWHFTTPLLLLDLALLVDADE

SEQ ID NO: 148 - protein, 34 aa, synthetic construct; identical to SEQ ID NO: 44

SEQ ID NO: 149 - protein, 33 aa, synthetic construct
AEQNPIYFARYADWLFTTPLLLLDLALLVDADE

SEQ ID NO: 150 - protein, 34 aa, synthetic construct; identical to SEQ ID NO: 46

SEQ ID NO: 151 - protein, 32 aa, synthetic construct
ADNNPWIYARYADLTTFPLLLLDLALLVDFDD

SEQ ID NO: 152 - protein, 32 aa, synthetic construct
ADNNPFIYARYADLTTWPLLLLDLALLVDFDD

SEQ ID NO: 153 - protein, 32 aa, synthetic construct
ADNNPFIYARYADLTTFPLLLLDLALLVDWDD

SEQ ID NO: 154 - protein, 32 aa, synthetic construct
ADNNPFPYARYADLTTWILLLLDLALLVDFDD

SEQ ID NO: 155 - protein, 32 aa, synthetic construct
ADNNPFIYAYRADLTTFPLLLLDLALLVDWDD

SEQ ID NO: 156 - protein, 32 aa, synthetic construct
ADNNPFIYATYADLRTFPLLLLDLALLVDWDD

SEQ ID NO: 157 - protein, 33 aa, synthetic construct
  source 1..33: note = Acetylated N-terminus, Amidated C terminus
TEDADVLLALDLLLLPTTFLWDAYRAWYPNQEA

SEQ ID NO: 158 - protein, 30 aa, synthetic construct
AEDQNPYWARYADWLFTTPLLLLDLALLVD

SEQ ID NO: 159 - protein, 30 aa, synthetic construct
AEDQNPYWARYADWLFTTPLLLLELALLVE

SEQ ID NO: 160 - protein, 28 aa, synthetic construct
AEDQNPYWRAYADLFTPLTLLDLLALWD

SEQ ID NO: 161 - protein, 26 aa, synthetic construct
ADDQNPWRAYLDLLFPTDTLLLDLLW

SEQ ID NO: 162 - protein, 30 aa, synthetic construct
ADDQNPWRAYLDLLFPTDTLLLDLLWDADE

SEQ ID NO: 163 - protein, 26 aa, synthetic construct
  MOD_RES 12: note = Gamma-carboxyglutamic acid
ADDQNPWRAYLXLLFPTDTLLLDLLW

SEQ ID NO: 164 - protein, 26 aa, synthetic construct
  MOD_RES 18: note = Gamma-carboxyglutamic acid
ADDQNPWRAYLDLLFPTXTLLLDLLW

SEQ ID NO: 165 - protein, 26 aa, synthetic construct
  MOD_RES 23: note = Gamma-carboxyglutamic acid
ADDQNPWRAYLDLLFPTDTLLLXLLW

SEQ ID NO: 166 - protein, 26 aa, synthetic construct
  MOD_RES 12: note = Gamma-carboxyglutamic acid
  MOD_RES 18: note = Gamma-carboxyglutamic acid
ADDQNPWRAYLXLLFPTXTLLLDLLW

SEQ ID NO: 167 - protein, 26 aa, synthetic construct
  MOD_RES 12: note = Gamma-carboxyglutamic acid
  MOD_RES 23: note = Gamma-carboxyglutamic acid
ADDQNPWRAYLXLLFPTDTLLLXLLW

SEQ ID NO: 168 - protein, 26 aa, synthetic construct
  MOD_RES 18: note = Gamma-carboxyglutamic acid
  MOD_RES 23: note = Gamma-carboxyglutamic acid
ADDQNPWRAYLDLLFPTXTLLLXLLW

SEQ ID NO: 169 - protein, 26 aa, synthetic construct
  MOD_RES 12: note = Gamma-carboxyglutamic acid
  MOD_RES 18: note = Gamma-carboxyglutamic acid
  MOD_RES 23: note = Gamma-carboxyglutamic acid
ADDQNPWRAYLXLLFPTXTLLLXLLW

SEQ ID NO: 170 - protein, 26 aa, synthetic construct
  MOD_RES 12: note = Alpha-aminoadipic acid
ADDQNPWRAYLXLLFPTDTLLLDLLW

SEQ ID NO: 171 - protein, 26 aa, synthetic construct
  MOD_RES 18: note = Alpha-aminoadipic acid
ADDQNPWRAYLDLLFPTXTLLLDLLW

SEQ ID NO: 172 - protein, 26 aa, synthetic construct
  MOD_RES 23: note = Alpha-aminoadipic acid
ADDQNPWRAYLDLLFPTDTLLLXLLW

SEQ ID NO: 173 - protein, 26 aa, synthetic construct
  MOD_RES 12: note = Alpha-aminoadipic acid
  MOD_RES 18: note = Alpha-aminoadipic acid
ADDQNPWRAYLXLLFPTXTLLLDLLW

SEQ ID NO: 174 - protein, 26 aa, synthetic construct
  MOD_RES 12: note = Alpha-aminoadipic acid
  MOD_RES 23: note = Alpha-aminoadipic acid
ADDQNPWRAYLXLLFPTDTLLLXLLW

SEQ ID NO: 175 - protein, 26 aa, synthetic construct
  MOD_RES 18: note = Alpha-aminoadipic acid
  MOD_RES 23: note = Alpha-aminoadipic acid
ADDQNPWRAYLDLLFPTXTLLLXLLW

SEQ ID NO: 176 - protein, 26 aa, synthetic construct
  MOD_RES 12: note = Alpha-aminoadipic acid
  MOD_RES 18: note = Alpha-aminoadipic acid
  MOD_RES 23: note = Alpha-aminoadipic acid
ADDQNPWRAYLXLLFPTXTLLLXLLW

SEQ ID NO: 177 - protein, 26 aa, synthetic construct
  MOD_RES 18: note = Alpha-aminoadipic acid
  MOD_RES 12: note = Gamma-carboxyglutamic acid
ADDQNPWRAYLXLLFPTXTLLLDLLW

SEQ ID NO: 178 - protein, 26 aa, synthetic construct
  MOD_RES 12: note = Gamma-carboxyglutamic acid
  MOD_RES 23: note = Alpha-aminoadipic acid
ADDQNPWRAYLXLLFPTDTLLLXLLW

SEQ ID NO: 179 - protein, 26 aa, synthetic construct
  MOD_RES 12: note = Gamma-carboxyglutamic acid
  MOD_RES 18: note = Gamma-carboxyglutamic acid
  MOD_RES 23: note = Alpha-aminoadipic acid
ADDQNPWRAYLXLLFPTXTLLLXLLW

SEQ ID NO: 180 - protein, 26 aa, synthetic construct
  MOD_RES 12: note = Alpha-aminoadipic acid
  MOD_RES 18: note = Gamma-carboxyglutamic acid
ADDQNPWRAYLXLLFPTXTLLLDLLW

SEQ ID NO: 181 - protein, 26 aa, synthetic construct
  MOD_RES 12: note = Alpha-aminoadipic acid
  MOD_RES 23: note = Gamma-carboxyglutamic acid
ADDQNPWRAYLXLLFPTDTLLLXLLW

SEQ ID NO: 182 - protein, 26 aa, synthetic construct
  MOD_RES 12: note = Gamma-carboxyglutamic acid
  MOD_RES 23: note = Gamma-carboxyglutamic acid
  MOD_RES 18: note = Alpha-aminoadipic acid
ADDQNPWRAYLXLLFPTXTLLLXLLW

SEQ ID NO: 183 - protein, 29 aa, synthetic construct
GEEQNPWLGAYLDLLFPLELLGLLELGLW

SEQ ID NO: 184 - protein, 28 aa, synthetic construct
ADDDDDDPWQAYLDLLFPTDTLLLDLLW

SEQ ID NO: 185 - protein, 26 aa, synthetic construct
AEEQNPWRAYLELLFPTETLLLELLW

SEQ ID NO: 186 - protein, 24 aa, synthetic construct
ADDQNPWARYLDWLFPTDTLLLDL

SEQ ID NO: 187 - protein, 23 aa, synthetic construct
DNNNPWRAYLDLLFPTDTLLLDW

SEQ ID NO: 188 - protein, 24 aa, synthetic construct
AEEQNPWARYLEWLFPTETLLLEL

SEQ ID NO: 189 - protein, 25 aa, synthetic construct
DDDDDDPWQAYLDLFPTDTLALDLW

SEQ ID NO: 190 - protein, 23 aa, synthetic construct
EEQQPWAQYLELLFPTETLLLEW

SEQ ID NO: 191 - protein, 23 aa, synthetic construct
EEQQPWRAYLELLFPTETLLLEW

SEQ ID NO: 192 - protein, 23 aa, synthetic construct
AEDQNPWARYADWLFPTTLLLLD

SEQ ID NO: 193 - protein, 23 aa, synthetic construct
AEEQNPWARYAEWLFPTTLLLLE

SEQ ID NO: 194 - protein, 21 aa, synthetic construct
AEDQNPWARYADLLFPTTLAW

SEQ ID NO: 195 - protein, 21 aa, synthetic construct
AEEQNPWARYAELLFPTTLAW

SEQ ID NO: 196 - protein, 36 aa, synthetic construct
DDDDDNPNYWARYANWLFTTPLLLLNGALLVEAEET

SEQ ID NO: 197 - protein, 36 aa, synthetic construct
DDDDDNPNYWARYAPWLFTTPLLLLPGALLVEAEET

SEQ ID NO: 198 - protein, 34 aa, synthetic construct; identical to SEQ ID NO: 46

SEQ ID NO: 199 - protein, 34 aa, synthetic construct; identical to SEQ ID NO: 44

SEQ ID NO: 200 - protein, 20 aa, synthetic construct
AEQNPIYWARYADWLFTTPL

SEQ ID NO: 201 - protein, 22 aa, synthetic construct
AEQNPIYFARYADLLFPTTLAW

SEQ ID NO: 202 - protein, 22 aa, synthetic construct
AEQNPIYWARYADLLFPTTLAF

SEQ ID NO: 203 - protein, 22 aa, synthetic construct
AEQNPIYWARYADLLFPTTLAW

SEQ ID NO: 204 - protein, 20 aa, synthetic construct
AEQNPIYFARYADWLFTTPL

SEQ ID NO: 205 - protein, 20 aa, synthetic construct
EDQNPWARYADLLFPTTLAW

SEQ ID NO: 206 - protein, 34 aa, synthetic construct
GLAGLAGLLGLEGLLGLPLGLLEGLWLGLELEGN

SEQ ID NO: 207 - protein, 32 aa, synthetic construct
EQNPIYILDLVFGLLFAVTSVDFLVQWDDAGD

SEQ ID NO: 208 - protein, 24 aa, synthetic construct
NLEGFFATLGGEIALWSLVVLAIE

SEQ ID NO: 209 - protein, 24 aa, synthetic construct
NNEGFFATLGGEIALWSDVVLAIE

SEQ ID NO: 210 - protein, 25 aa, synthetic construct
DNNEGFFATLGGEIPLWSDVVLAIE

SEQ ID NO: 211 - protein, 36 aa, synthetic construct
  source 1..36: note = Acetylated N-terminus
AEQNPIYWARYADWLFTTPLLLLDLALLVDADEGCT

SEQ ID NO: 212 - protein, 28 aa, synthetic construct
ADQDNPWRAYLDLLFPTDTLLLDLLWCA

SEQ ID NO: 213 - protein, 28 aa, synthetic construct
ADQDNPWRAYLDLLFPTDTLLLDLLWKA

SEQ ID NO: 214-217 - skipped (4 entries)

SEQ ID NO: 218 - protein, 28 aa, synthetic construct
ADQDNPWRAYLDLLFPTDTLLLDLLWCG

SEQ ID NO: 219 - protein, 28 aa, synthetic construct
ADDQNPWRAYLDLLFPTDTLLLDLLWCG

SEQ ID NO: 220 - protein, 28 aa, synthetic construct
  VARIANT 27: note = X is a functional group for conjugation purposes, selected from lysine (Lys), cysteine (Cys), Azido-containing amino acid or other modified amino acids
ADDQNPWRAYLDLLFPTDTLLLDLLWXG

SEQ ID NO: 221 - protein, 29 aa, synthetic construct
AKDDQNPWRAYLDLLFPTDTLLLDLLWCG